(19) United States
(12) Patent Application Publication
(10) Pub. No.: US 2007/0249552 A1
(43) Pub. Date: Oct. 25, 2007

(54) Compositions and Methods for Sirna Inhibition of Primate Polyomavirus Genes

(76) Inventors: Kamel Khalili, Merion, PA (US); Jennifer Gordon, Philadelphia, PA (US)

Correspondence Address: NATH & ASSOCIATES, 112 South West Street, Alexandria, VA, 22314, US

(21) Appl. No.: 11/596,479

(22) PCT Filed: May 12, 2005
(86) PCT No.: PCT/US05/16597

§ 371 Date: Nov. 13, 2006

Related U.S. Application Data
(60) Provisional application No. 60/570,345, filed on May 12, 2004.

(51) Int. Cl. A61K 31/713; A61K 31/7105; C07H 21/02; C12N 15/85; C12N 15/86

(52) U.S. Cl. 514/44; 435/320.1; 536/23.1

(57) ABSTRACT

RNA interference using small interfering RNAs which are specific for mRNA produced from the JCV agnoprotein and large T antigen genes inhibits expression of these and other primate polyomavirus genes. Primate polyomavirus infection, and diseases which are associated with primate polyomavirus infection, can be treated by administering the small interfering RNAs.

### FIELD OF THE INVENTION

This invention relates to the regulation of primate polyomavirus gene expression, such as human neurotropic polyomavirus JCV gene expression, by small interfering RNA. In particular, primate polyomavirus infection and diseases associated with primate polyomavirus infection can be treated.

### BACKGROUND OF THE INVENTION

Progressive multifocal leukoencephalopathy (PML) is a fatal demyelinating disease of the central nervous system (CNS) which results from reactivation of the latent polyomavirus, JCV, and its productive replication in glial cells of the human brain (Berger and Concha, 1995; Clifford and Major, 2001). Once a rare disease primarily seen in patients with impaired immune systems due to lymphoproliferative and myeloproliferative disorders, PML has become one of the major neurologic problems among patients with acquired immunodeficiency syndrome (AIDS) (Cinque et al, 2003). It has been reported that between 4% and 8% of AIDS patients exhibit signs of PML, and JCV has been detected in cerebrospinal fluid (CSF) of affected patients, suggesting active replication of the virus in the brain (Berger and Concha, 1995; Clifford and Major, 2001).

The histological hallmarks of PML include multifocal demyelinated lesions with enlarged hyperchromatic nuclei in oligodendrocytes and enlarged bizarre astrocytes with lobulated hyperchromatic nuclei within white matter tracts of the brain (Walker and Padgett, 1983; Cinque et al, 2003). Although in some instances, atypical features that include a unifocal pattern of demyelination and involvement of the gray matter have been reported (Sweeney et al., 1994; for review see Cinque et al., 2003 JNV). Earlier observations from in vitro cell culture studies and in vivo evaluation of JCV in clinical samples led to early assumptions that oligodendrocytes and astrocytes are the only cells which support productive viral infection (Frisque and White, 1992; Gordon and Khalili, 1998). Accordingly, molecular studies have provided evidence for cell type-specific transcription of the viral early genome in cells derived from the CNS (Raj and Khalili, 1995). However, subsequent studies have shown low, but detectable levels of the JCV gene expression in non-neural cells including B cells and noticeably high level production of the viral early protein in several neural and non-neural tumor cells in humans (Gordon and Khalili, 1998; Khalili et al, 2003).

In accord with the other polyomaviruses, JCV is a small DNA virus whose genome can be divided into three regions that encompass the transcription control region, the genes responsible for the expression of the viral early protein, T-antigen, and the viral late proteins, VP1, VP2, and VP3. In addition, the late genome is also responsible for production of the viral auxiliary protein, Agnoprotein. T-antigen expression is pivotal for initiation of the viral lytic cycle, as this protein stimulates transcription of the late genes, and induces the process of viral DNA replication (Frisque and White, 1992). Recent studies have ascribed an important role for Agnoprotein in transcription and replication of JCV, as inhibition of its production significantly reduces viral gene expression and replication (Safak et al, unpublished observations). Furthermore, agnoprotein dysregulates the cell cycle by altering the expression of several cyclins and their associated kinases (Darbinyan et al, 2002).

Thus far, there are no effective therapies for the suppression of JCV replication and treatment of PML. Cytosine arabinoside (AraC) had been tested for the treatment of PML patients and the outcome, in some instances, revealed remission of JCV-associated demyelination (for review see Aksamit et al., 2001 JNV 7:386 and references within). Reports from the AIDS Clinical Trial Group (ACTG) Organized Trial 243, however, have suggested that there is no difference in the survival of HIV-1 infected patients with PML compared to the control population (Hall et al., 1998). Although, in other reports, it has been suggested that the failure of AraC in the ACTG trial may have been due to insufficient delivery of the AraC via the intravenous and intrathecal routes (Levy et al., 2001). Based on in vitro studies showing the ability of inhibitors of topoisomerase to suppress JCV DNA replication (Kerr et al., 1993), the topoisomerase inhibitor, topotecan was used in the treatment of AIDS/PML patients and results suggested that topotecan treatment may be associated with decreased lesion size and prolonged survival (Royal et al., 2003).

In addition, JCV infection has been linked to various tumors of central nervous system (CNS) origin, including medullablastoma, glioblastoma, and others. Del Valle L et al., (2001a), supra; Khalili K (1999), supra. Examination of T-antigen expression in the CNS tumor tissue revealed that not all tumor cells express T-antigen. Evaluation of these tumors for other viral proteins showed a substantial level of agnoprotein in tumors containing the JCV genome. DeValle L et al. (2002), J. Nat. Cancer Inst. 94(4): 267-273.

The importance of agnogene expression in brain tumor cells is unknown. One hypothesis holds that interactions of T-antigen and agnoprotein with each other, and with endogenous cellular proteins, can modulate the growth rate of tumor cells. Nevertheless, it appears from the studies discussed above that JCV agnoprotein is involved in the development and growth of some CNS neoplasms.

RNA interference (hereinafter “RNAi”) is a method of post-transcriptional gene regulation that is conserved throughout many eukaryotic organisms. RNAi is induced by short (i.e., <30 nucleotide) double stranded RNA (“dsRNA”) molecules which are present in the cell (Fire A et al. (1998), Nature 391: 806-811). These short dsRNA molecules, called “short interfering RNA” or “siRNA,” cause the destruction of messenger RNAs (“mRNAs”) which share sequence homology with the siRNA to within one nucleotide resolution (Elbashir S M et al. (2001), Genes Dev, 15: 188-200). It is believed that the siRNA and the targeted mRNA bind to an “RNA-induced silencing complex” or “RISC”, which cleaves the targeted mRNA. The siRNA is apparently recycled much like a multiple-turnover enzyme, with 1 siRNA molecule capable of inducing cleavage of approximately 1000 mRNA molecules. siRNA-mediated RNAi degradation of an mRNA is therefore more effective than currently available technologies for inhibiting expression of a target gene.

Elbashir S M et al. (2001), supra, has shown that synthetic siRNA of 21 and 22 nucleotides in length, and which have short 3′ overhangs, are able to induce RNAi of target mRNA in a Drosophila cell lysate. Cultured mammalian cells also exhibit RNAi degradation with synthetic siRNA (Elbashir S M et al. (2001) Nature, 411: 494-498), and RNAi degradation induced by synthetic siRNA has recently been shown in living mice (McCaffrey A P et al. (2002), Nature, 418: 38-39; Xia H et al. (2002), Nat. Biotech. 20: 1006-1010). The therapeutic potential of siRNA-induced RNAi degradation has been demonstrated in several recent in vitro studies, including the siRNA-directed inhibition of HIV-1 infection (Novina C D et al. (2002), Nat. Med. 8: 681-686) and reduction of neurotoxic polyglutamine disease protein expression (Xia H et al. (2002), supra).

What is needed, therefore, are agents and methods which selectively inhibit expression of primate polyomavirus genes, in particular the agnoprotein and large T antigen genes, in catalytic or sub-stoichiometric amounts, in order to effectively decrease or block JCV infection and replication, and to treat diseases associated with JCV infection.

### SUMMARY OF THE INVENTION

The present invention is directed to siRNA which specifically target and cause RNAi-induced degradation of mRNA from primate polyomavirus genes, in particular from the agnoprotein and large T antigen genes of JCV. These siRNA degrade agnoprotein and large T antigen mRNA in substoichiometric amounts. The siRNA compounds and compositions of the invention can be used to inhibit JCV, BKV and/or SV40 infection and replication, and treat diseases associated with JCV, BKV and/or SV40 infection. In particular, the siRNA of the invention are useful for treating cancer or progressive multifocal leukoencephalopathy (“PLM”).

Thus, the invention provides an isolated siRNA which targets JCV agnoprotein gene or large T antigen gene mRNA, or an alternative splice form or mutant thereof. The siRNA comprises a sense RNA strand and an antisense RNA strand which form an RNA duplex. The sense RNA strand comprises a nucleotide sequence substantially identical to a target sequence of about 19 to about 25 contiguous nucleotides in the target mRNA. The mRNA produced from BKV and/or SV40 genes can have sequences in common with JCV mRNA; thus, the siRNA of the invention that target and degrade JCV mRNA also target and degrade BKV and/or SV40 mRNA, when the BKV and/or SV40 mRNA contains a target sequence in common with the JCV mRNA.

The invention also provides recombinant plasmids and viral vectors which express the siRNA of the invention, as well as pharmaceutical compositions comprising the siRNA of the invention and a pharmaceutically acceptable carrier.

The invention further provides a method of inhibiting expression of JCV agnoprotein gene and/or large T antigen gene mRNA, or alternative splice forms or mutants thereof, comprising administering to a subject an effective amount of one or more of the siRNA of the invention such that the target mRNA is degraded.

The invention further provides a method of inhibiting expression of JCV VP1 protein, comprising administering to a subject an effective amount of one or more of the siRNA targeted to JCV agnoprotein gene and/or large T antigen gene mRNA, or alternative splice forms or mutants thereof.

The invention further provides a method of inhibiting JCV infection or replication in a subject, comprising administering to a subject an effective amount of an siRNA targeted to JCV agnoprotein gene and/or large T antigen gene mRNA, or alternative splice forms or mutants thereof.

The invention further provides a method of treating diseases associated with JCV infection, for example cancer or PML, comprising administering to a subject in need of such treatment an effective amount of an siRNA targeted to JCV agnoprotein gene and/or large T antigen gene mRNA, or alternative splice forms or mutants thereof.

### BRIEF DESCRIPTION OF THE FIGURES

FIG. 1. JCV T-antigen siRNA decreases expression of JCV proteins in transiently transfected and infected primary human astrocytes. Primary human fetal astrocytes were prepared as described previously and were seeded onto 6 well plates at a density of 500,000 cells/well (Radhakrishnan et al, 2003). For transient transfections, cells were transfected using FuGENE 6 with plasmid expressing JCV T-antigen (Kerr et al, 1993). The following day, the cells were transfected with double-stranded 21-bp siRNA for JCV T-antigen targeting nt 4256-4276 of the Mad-1 isolate of JCV, sense strand 5′-AAGUCUUUAGGGUCUUCUACCUdTdT-3′. The siRNAs were prepared as double stranded, 2′-deprotected and desalted oligonucleotide and were utilized according to the manufacturer's directions (Dharmacon). For transfection of siRNA, 100 μmol of siRNA was mixed with 3 ul of Oligofectamine (Invitrogen), diluted in OptiMEM (Invitrogen) and incubated with the dell cultures for 4 h at 37° C. in serum- and antibiotic-free conditions. After transfection, the cells were fed with serum-containing media without removing the siRNA transfection mixture. A. Whole cell extracts prepared from transfected astrocytes 24 h after siRNA treatment were analyzed by Western blotting for the presence of T-antigen (pAb416, Oncogene Science) and the unrelated protein, grb-2 (upper and lower panels, respectively). B. In parallel, samples transfected with 1.0 ug of JCV T-antigen expression plasmid along with 0.5 ug of a luciferase reporter construct containing the JCV late promoter (Mad-1 strain) were harvested 24 h after siRNA treatment, and luciferase activity was determined according to the manufacturer's directions (Promega luciferase assay system). Activity is presented as a difference in fold change with the background activity of the JCV late promoter arbitrarily set a 1 from 4 experiments. Standard deviations are indicated by error bars. C. Primary astrocytes were infected with JCV Mad-4 strain at an M.O.I. of 1 in serum-free media for 3 h at 37° C. Uninfected and infected cells were then transfected with T-antigen siRNA at days 1, 5, and 10 post-infection and were harvested at day 15. Western blotting was performed on whole cell extracts for the presence of JCV early and late proteins, T-antigen (pAb416, Oncogene Research Products), Agnoprotein (Del Valle et al, 2001a), and VP1 (pAb597, kindly provided by Walter Atwood, Brown University) as well as the cellular protein, grb-2 (pAb81, BD Biosciences). Proteins were visualized using horseradish peroxidase conjugated secondary antibodies and the ECL-Plus system (Amersham).

FIG. 2. JCV Agnoprotein siRNA decreases Agnogene expression as well as other viral proteins in primary human astrocytes. Primary human fetal astrocyte preparation, transient transfections, siRNA treatment, and Western blotting were performed as described in the text and the legend to FIG. 1. Cells were transfected with a plasmid containing the JCV Agnoprotein fused to YFP (Darbinyan et al, 2002). JCV Agnoprotein siRNA targeted nt 324-342 of the Mad-1 isolate of JCV, sense strand 5′-AACCUGGAGUGGAACUAAAdTdT-3′ while non-specific siRNA (ns siRNA) targeted nt 435-453 of the Dunlop strain of BKV, sense strand 5′-AACCUGGACUGGAACAAAAdTdT-3′. The two base pair mismatches between JCV and BKV Agnoprotein sequences are underlined. A. Whole cell extracts prepared from transfected astrocytes 24 h after treatment with specific or non-specific siRNAs were analyzed by Western blotting for the presence of Agnoprotein and the unrelated cellular factor, grb-2 (upper and lower panels, respectively). B. Primary astrocytes, uninfected and infected with the JCV Mad-4 strain were then transfected with JCV Agnoprotein or non-specific BKV Agnoprotein siRNA at days 1, 5, and 10 post-infection and were harvested at day 15. Western blotting was performed on whole cell extracts for the presence of JCV T-antigen, Agnoprotein, and VP 1 as well as the cellular protein, grb-2.

FIG. 3. Treatment with siRNAs targeting JCV T-antigen and Agnoprotein abrogate their expression in primary human astrocytes as well as of the JCV late protein, VP1, in infected cells. Primary human fetal astrocyte preparation, transient transfections, siRNA treatment, and Western blotting were performed as described in the text and the legend to FIG. 1. A. Whole cell extracts from astrocytes transfected with expression plasmids for JCV T-antigen, YFP-Agnoprotein, or both were prepared from the cultures 24 h after siRNA treatment and were analyzed by Western blotting for the presence of T-antigen and Agnoprotein as well as for the unrelated grb-2. B. Astrocyte cultures were infected with the JCV Mad-4 strain of JCV and were then transfected with siRNA targeting JCV T-antigen, Agnoprotein, or both at days 1, 5, and 10 post-infection. Western blotting was performed on whole cell extracts harvested at day 15 post-infection for the presence of JCV T-antigen and Agnoprotein, as well as the viral late protein, VP 1, and the cellular protein, grb-2.

FIG. 4. Immunocytochemistry of primary astrocytes infected with JCV reveals alterations in JCV protein levels upon JCV T-antigen and Agnoprotein siRNA treatment. Cells were infected with JCV and treated at days 1, 5, and 10 post-infection with JCV T-antigen siRNA, JCV Agnoprotein siRNA, or both. Cells were also treated with BKV siRNA. Cells were subcultured and plated onto poly-L-lysine coated chamber slides (Falcon) on day 13 and were fixed on day 15 post-infection with ice-cold acetone for 3 min. Viral proteins were detected by immunocytochemistry as described previously (Radhakrishnan et al, 2003) using the same primary antibodies as for Western blot analysis (see legend to FIG. 1). Proteins were visualized with fluorescein-conjugated secondary antibodies.

FIG. 5 shows the alignment of nucleic acid sequences from JCV, BK virus (BKV) and SV40 agnoprotein genes. Identical bases are shown in grey; non-identical bases are shown in white.

FIGS. 6a to 6g show the alignment of nucleic acid sequences from JCV, BKV and SV40 large T antigen genes. Identical bases are shown in grey; non-identical bases are shown in white.

### DETAILED DESCRIPTION OF THE INVENTION

Unless otherwise indicated, all nucleic acid sequences herein are given in the 5′ to 3′ direction. Also, all deoxyribonucleotides in a nucleic acid sequence are represented by capital letters (e.g., deoxythymidine is “T”), and ribonucleotides in a nucleic acid sequence are represented by lower case letters (e.g., uridine is “u”).

The human polyomavirus, JCV, encodes two regulatory proteins at the early (T-antigen) and the late (agnoprotein) phase of viral infection whose activities are important for the production of the viral capsid proteins and dysregulation of several host factors and their functions. Here we designed and utilized an RNA interference strategy via siRNAs that target expression of T-antigen and Agnoprotein in human astrocytic cells. Treatment of the cells with specific siRNA oligonucleotides targeting a conserved region of T-antigen, nt 4256 to 4276 (Mad-1 strain), caused greater than 50% decline in the level of T-antigen and its transcriptional activity upon the viral capsid genes. Similarly, a single siRNA that aimed at nt 324 to 342 of Agnoprotein noticeably reduced viral early and late protein production. Combined treatment of the infected cells with both T-antigen and Agnoprotein siRNAs completely abrogated viral capsid protein production indicative of their ability to effectively halt multiplication of virus in the infected cells. These observations provide a new avenue for possible treatment of patients with the JCV-induced demyelinating disease, progressive multifocal leukoencephalopathy (PML).

Compositions and methods comprising siRNA targeted to JCV agnoprotein gene or large T antigen gene mRNA are advantageously used to inhibit JCV infection and replication, in particular for the treatment of JCV-associated diseases. The siRNA of the invention are believed to cause the RNAi-mediated degradation of these mRNAs, so that the protein products of the JCV agnoprotein gene or large T antigen gene are not produced or are produced in reduced amounts. Because JCV agnoprotein gene and the large T antigen gene are required for the JCV life-cycle, the siRNA-mediated degradation of JCV agnoprotein gene or large T antigen gene mRNA inhibits JCV infection and thus any pathology related to such infection; for example JCV-related demyelinating disease.

As used herein, siRNA which is “targeted to the JCV agnoprotein gene or large T antigen gene mRNA” means siRNA in which a first strand of the duplex is substantially identical to the nucleotide sequence of a portion of the JCV agnoprotein gene or large T antigen gene mRNA sequence. It is understood that the second strand of the siRNA duplex is complementary to both the first strand of the siRNA duplex and to the same portion of the JCV agnoprotein gene or large T antigen gene mRNA.

The invention therefore provides isolated siRNA comprising short double-stranded RNA from about 17 nucleotides to about 29 nucleotides in length, preferably from about 19 to about 25 nucleotides in length, that are targeted to the target mRNA. The siRNA's comprise a sense RNA strand and a complementary antisense RNA strand annealed together by standard Watson-Crick base-pairing interactions (hereinafter “base-paired”). As is described in more detail below, the sense strand comprises a nucleic acid sequence which is substantially identical to a target sequence contained within the target mRNA.

As used herein, a nucleic acid sequence “substantially identical” to a target sequence contained within the target mRNA is a nucleic acid sequence which is identical to the target sequence, or which differs from the target sequence by one or more nucleotides. Sense strands of the invention which comprise nucleic acid sequences substantially identical to a target sequence are characterized in that siRNA comprising such sense strands induce RNAi-mediated degradation of mRNA containing the target sequence. For example, an siRNA of the invention can comprise a sense strand which comprises nucleic acid sequences that differ from a target sequence by one, two or three or more nucleotides, as long as RNAi-mediated degradation of the target mRNA is induced by the siRNA.

The sense and antisense strands of the present siRNA can comprise two complementary, single-stranded RNA molecules or can comprise a single molecule in which two complementary portions are base-paired and are covalently linked by a single-stranded “hairpin” area. Without wishing to be bound by any theory, it is believed that the hairpin area of the latter type of siRNA molecule is cleaved intracellularly by the “Dicer” protein (or its equivalent) to form a siRNA of two individual base-paired RNA molecules (see Tuschl, T. (2002), szipra). As described below, the siRNA can also contain alterations, substitutions or modifications of one or more ribonucleotide bases. For example, the present siRNA can be altered, substituted or modified to contain one or more deoxyribonucleotide bases.

As used herein, “isolated” means synthetic, or altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered. By way of example, siRNA which are produced inside a cell by natural processes, but which are produced from an “isolated” precursor molecule, are themselves “isolated” molecules. Thus, an isolated dsRNA can be introduced into a target cell, where it is processed by the Dicer protein (or its equivalent) into isolated siRNA.

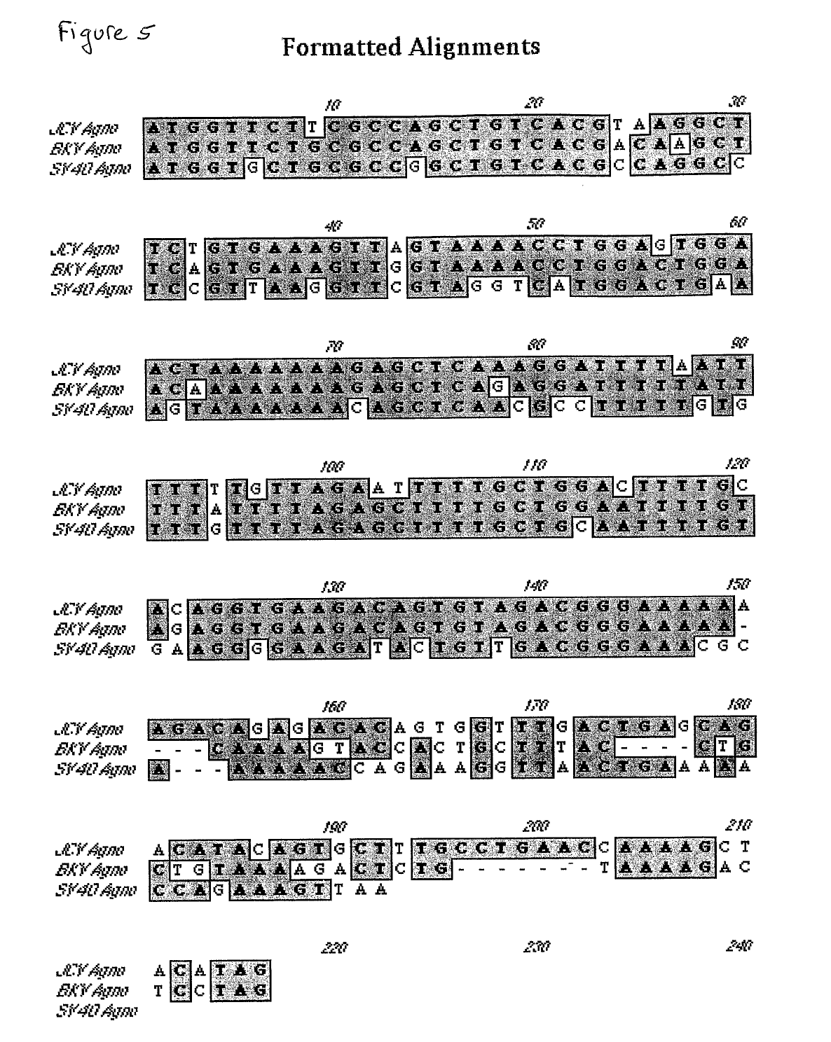

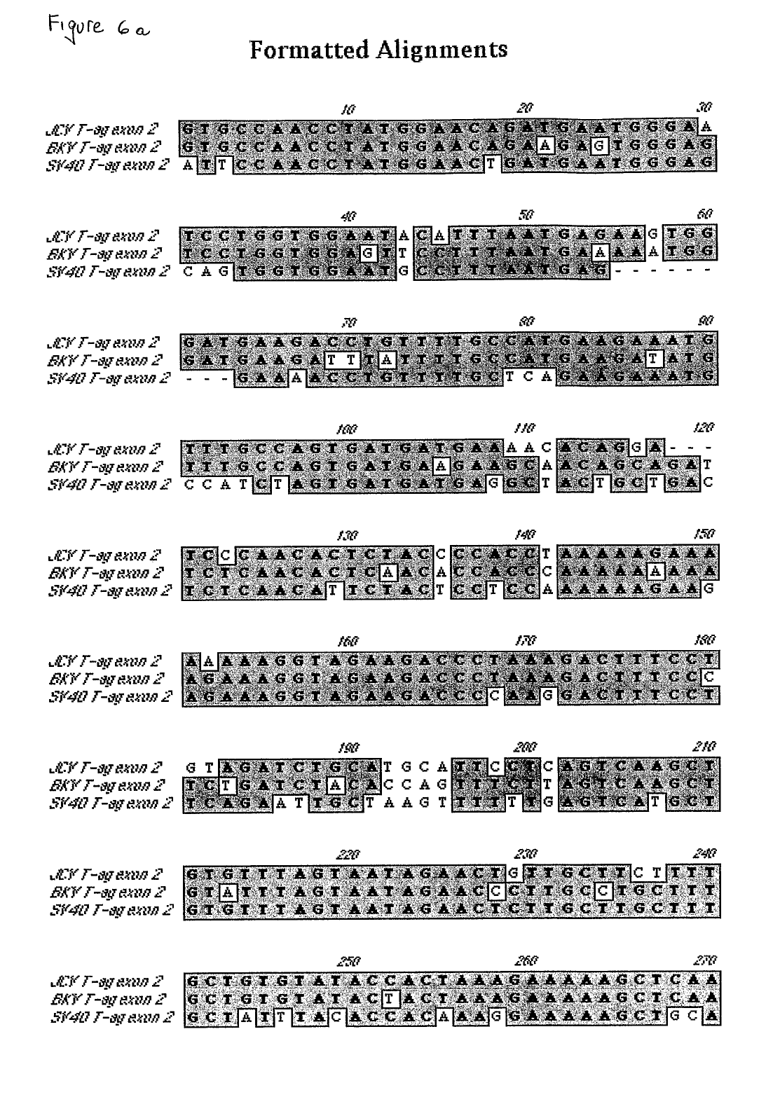

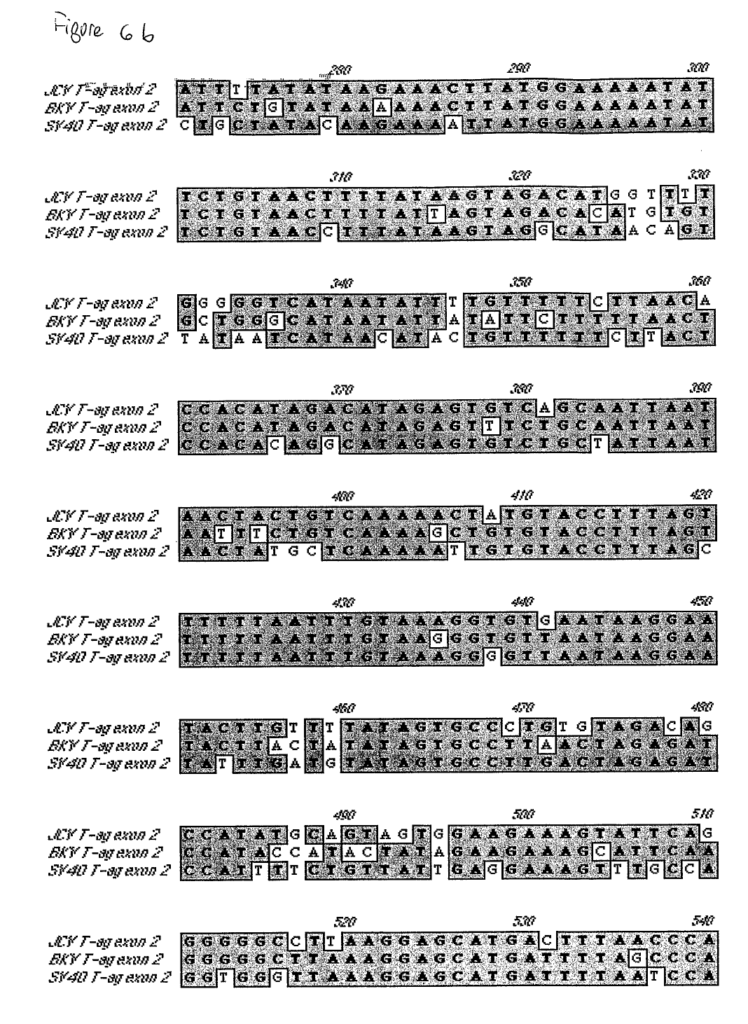

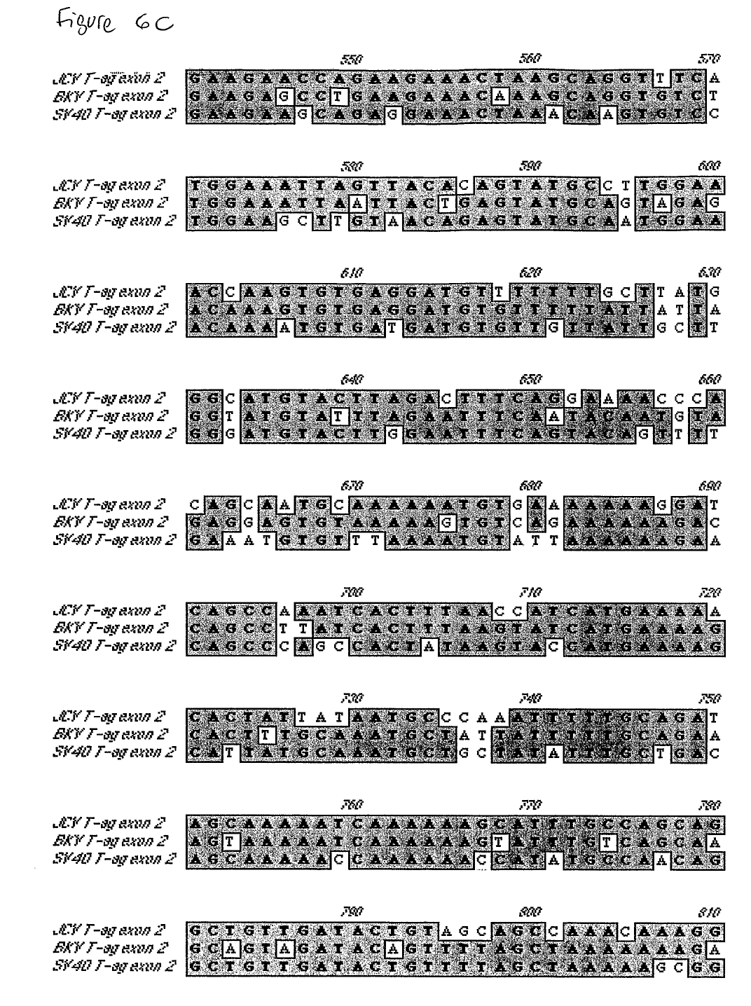

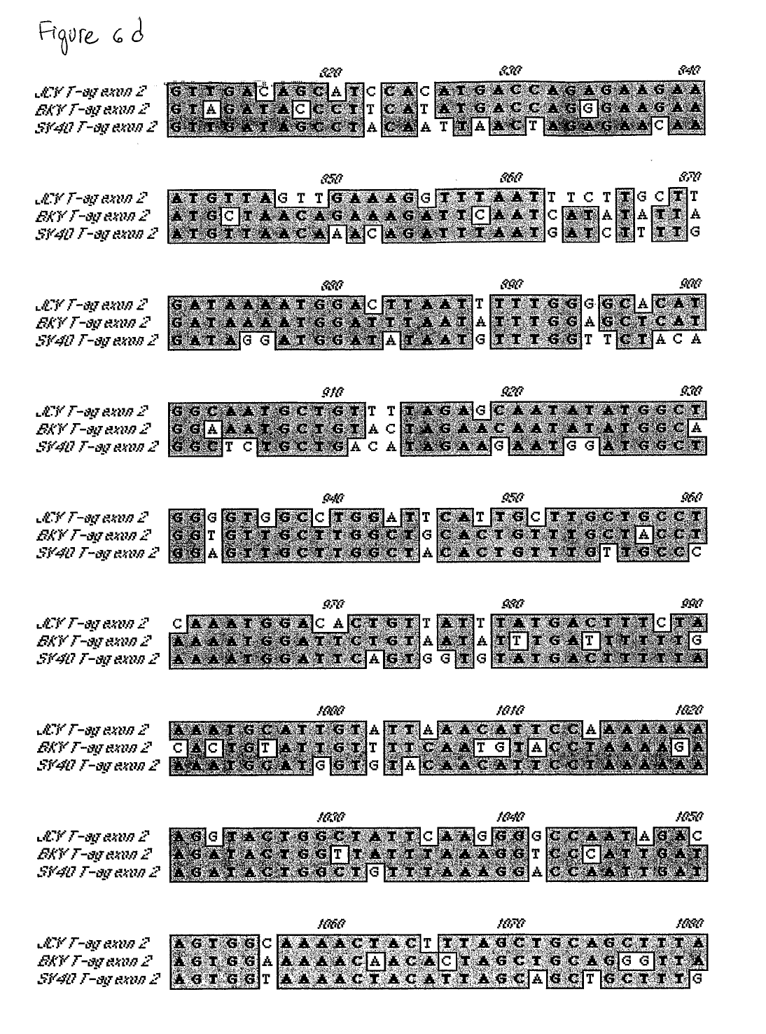

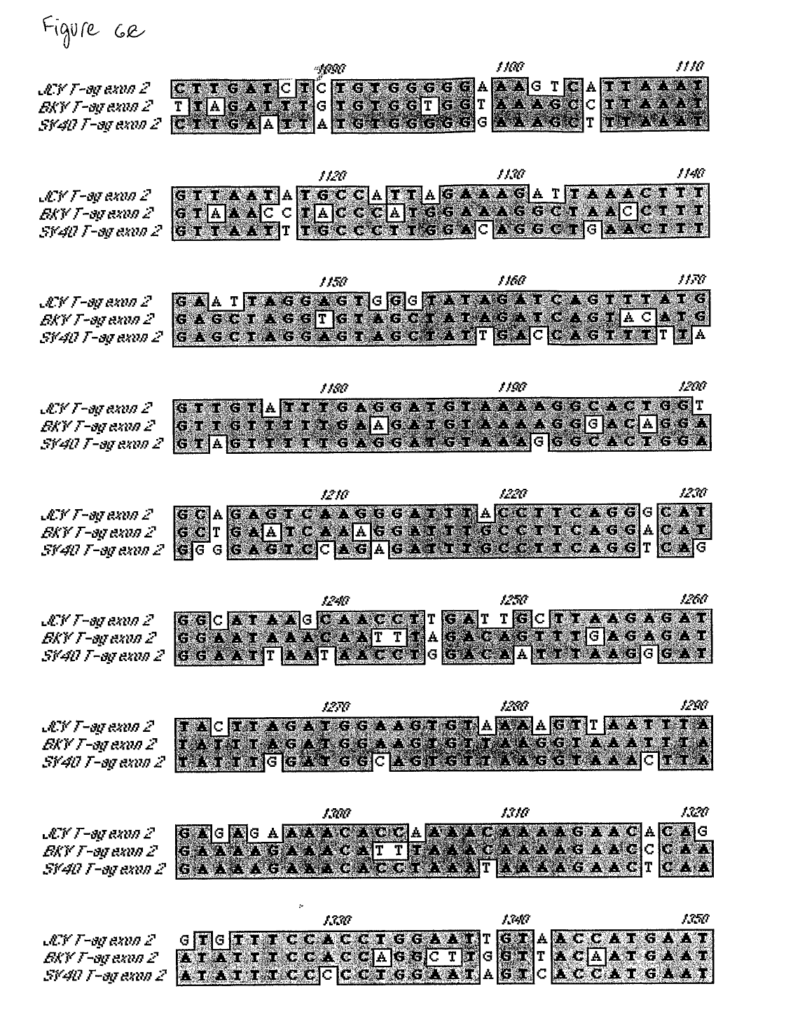

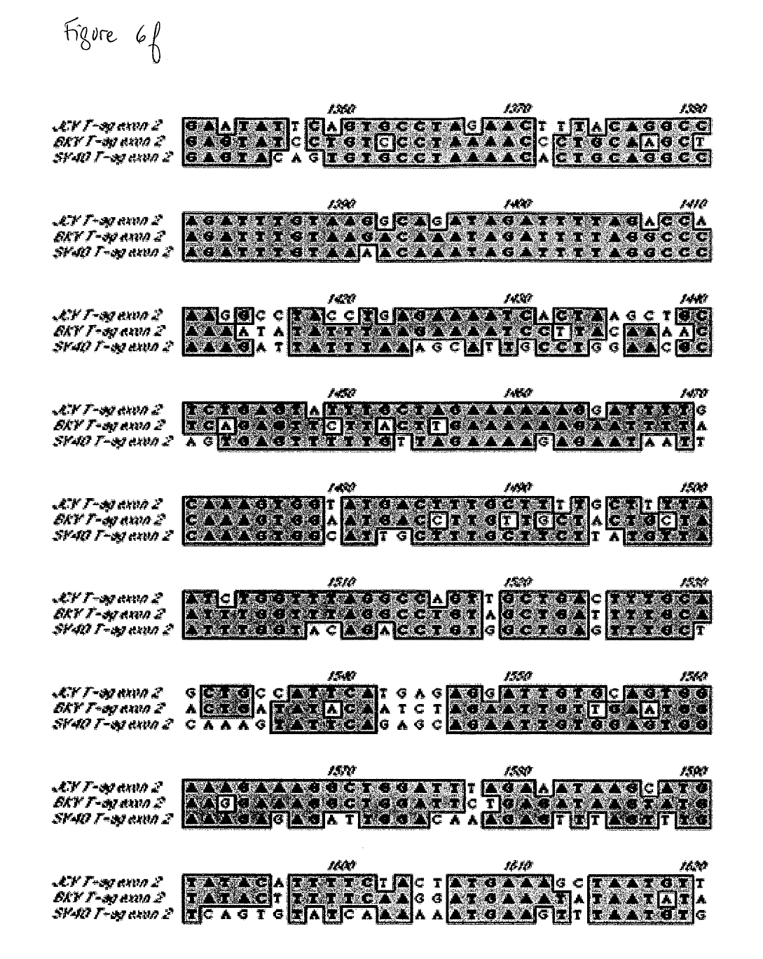

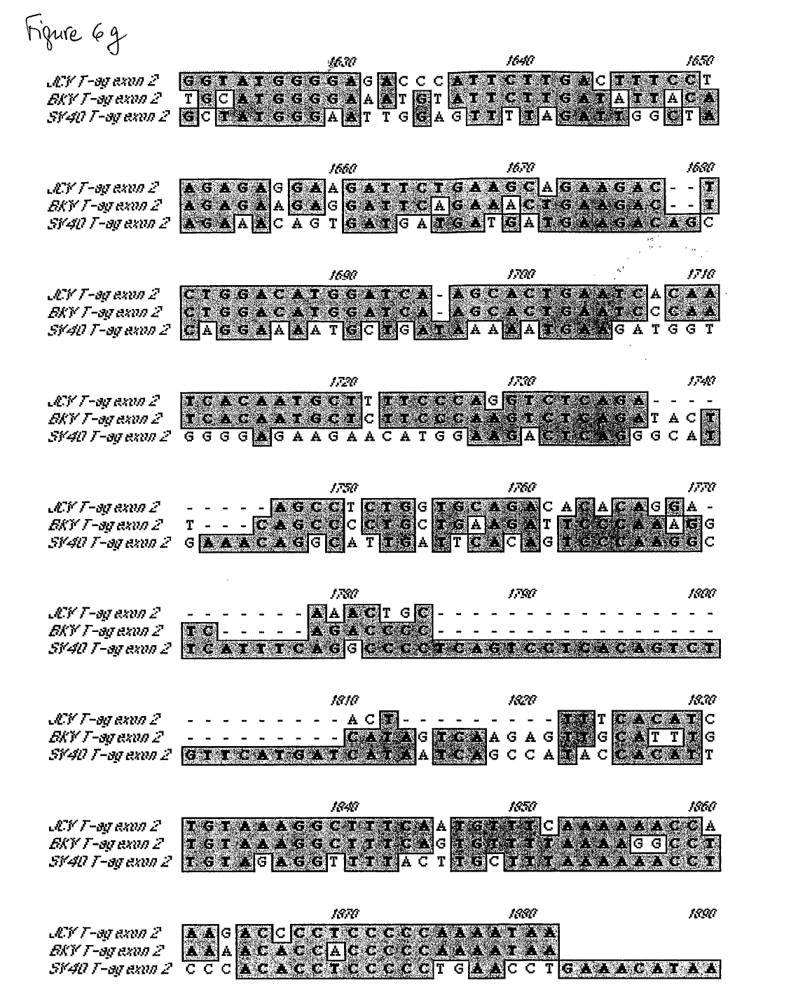

As used herein, “target mRNA” means JCV agnoprotein gene or large T antigen gene mRNA, or mutant or alternative splice forms of JCV agnoprotein gene or large T antigen gene mRNA. It is understood that genes from other primate polyomaviruses (such as BKV or SV40) can contain nucleotide sequences in common with JCV genes. Thus, mRNA produced from these other primate polyomavirus genes, which contain nucleotide sequences in common with JCV mRNA (see e.g., FIGS. 5 and 6), are considered “target mRNA” for purposes of the present invention. As used herein, a “primate polyomavirus” is any polyomavirus which infects a primate, in particular a human, and includes JCV, BKV and SV40.

Agnoprotein gene mRNA and large T antigen gene mRNA from any species and strain of primate polyomavirus can be used in the practice of the present invention. Preferred agnoprotein gene and large T antigen gene mRNA are those derived from JCV. The nucleic acid sequence encoding the JCV agnoprotein gene mRNA is given in SEQ ID NO: 1 as the cDNA equivalent.

Other JCV agnoprotein gene mRNAs known in the art include those disclosed (as the cDNA equivalents) in GenBank records Accession No. AF187236 (SEQ ID NO: 2), Accession No. AF281599 (SEQ ID NO: 3), Accession No. AF187234 (SEQ ID NO: 4), Accession No. AF295737 (SEQ ID NO: 5) and Accession No. AF295739 (SEQ ID NO: 6), the entire disclosures of which are herein in incorporated by reference.

Agnoprotein from human BK polyomavirus (BKV) strains, or from SV40 polyomavirus strains, are also known. Examples of BKV agnoproteins gene mRNAs are given (as the cDNA equivalents) in GenBank record Accession Nos. M23122 (SEQ ID NO: 7) and D00678 (SEQ ID NO: 8), the disclosures of which are herein incorporated by reference. An example of SV40 agnoprotein is given (as the cDNA sequence) in GenBank record Accession No. M99359 (SEQ ID NO: 9), the disclosure of which is herein incorporated by reference.

The mRNA transcribed from the JCV agnoprotein and large T antigen genes can also be analyzed for alternative splice forms using techniques well-known in the art. Such techniques include reverse transcription-polymerase chain reaction (RT-PCR), northern blotting and in-situ hybridization. Techniques for analyzing mRNA sequences are described, for example, in Busting SA (2000), J. Mol. Endocrinol. 25: 169-193, the entire disclosure of which is herein incorporated by reference. Representative techniques for identifying alternatively spliced mRNAs are also described below.

For example, databases that contain nucleotide sequences related to a given disease gene can be used to identify alternatively spliced mRNA. Such databases include GenBank and Embase. An mRNA or gene sequence from the JCV agnoprotein and large T antigen genes can be used to query such a database to determine whether ESTs representing alternatively spliced mRNAs have been found.

A technique called “RNAse protection” can also be used to identify alternatively spliced JCV agnoprotein and large T antigen gene mRNAs. RNAse protection involves translation of a gene sequence into synthetic RNA, which is hybridized to RNA derived from other cells; for example, cells which are induced to express agnoprotein and large T antigen. The hybridized RNA is then incubated with enzymes that recognize RNA:RNA hybrid mismatches. Smaller than expected fragments indicate the presence of alternatively spliced mRNAs. The putative alternatively spliced mRNAs can be cloned and sequenced by methods well known to those skilled in the art.

RT-PCR can also be used to identify alternatively spliced JCV agnoprotein and large T antigen gene mRNAs. In RT-PCR, mRNA from cells known or induced to express JCV agnoprotein and large T antigen is converted into cDNA by the enzyme reverse transcriptase, using methods within the skill in the art. The entire coding sequence of the cDNA is then amplified via PCR using a forward primer located in the 3′ untranslated region, and a reverse primer located in the 5′ untranslated region. The amplified products can be analyzed for alternative splice forms, for example by comparing the size of the amplified products with the size of the expected product from normally spliced mRNA, e.g., by agarose gel electrophoresis. Any change in the size of the amplified product can indicate alternative splicing.

The mRNA produced from mutant JCV agnoprotein and large T antigen genes can also be readily identified with the techniques described above for identifying JCV agnoprotein and large T antigen gene mRNA alternative splice forms. As used herein, “mutant” JCV agnoprotein and large T antigen genes or mRNA include JCV agnoprotein and large T antigen genes or mRNA which differ in sequence from the JCV agnoprotein and large T antigen gene sequences set forth herein. Thus, allelic forms of the JCV agnoprotein and large T antigen genes, and the mRNA produced from them, are considered “mutants” for purposes of this invention. See, e.g., the JCV agnoprotein gene mRNA described in Jobes D V et al. (1999), J. Human Virol. 2(6): 350-358, the disclosure of which is herein incorporated by reference which encodes an agnoprotein with a 7-amino acid deletion in the C-terminal region.

It is understood that JCV agnoprotein gene and large T antigen gene mRNA may contain target sequences in common with its respective alternative splice forms or mutants, or in common with analogous genes from different strains or species of primate polyomaviruses. A single siRNA comprising such a common targeting sequence can therefore induce RNAi-mediated degradation of those different mRNAs which contain the common targeting sequence. For example, certain siRNA of the invention, which are targeted to JCV gene sequences, also target BKV and SV40 gene sequences. An siRNA of the invention that targets BKV sequences can be used to treat polyomavirus associated nephropathy (PVN), which occurs in kidney transplant recipients. PVN is a leading cause of graft loss and there are currently no treatments. An siRNA of the invention that targets SV40 gene sequences can be used to treat SV40-induced cancers, including mesothelioma.

For example, the nucleic acid sequence alignments of the JCV, BKV and SV40 agnoprotein genes, which indicate the regions of base sequence identity and non-identity, are shown in FIG. 5. Likewise, the nucleic acid sequence alignment of JCV, BKV and SV40 large T antigen genes, which indicate the regions of base sequence identity and non-identity, are shown in FIG. 6.

The siRNA of the invention can comprise partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA; modifications that make the siRNA resistant to nuclease digestion (e.g., the use of 2′-substituted ribonucleotides or modifications to the sugar-phosphate backbone); or the substitution of one or more nucleotides in the siRNA with deoxyribonucleotides. siRNA which are exposed to serum, lachrymal fluid or other nuclease-rich environments, or which are delivered topically (e.g., by eyedropper), are preferably altered to increase their resistance to nuclease degradation. For example, siRNA which are administered intravascularly or topically to the eye can comprise one or more phosphorothioate linkages.

One or both strands of the siRNA of the invention can also comprise a 3′ overhang. As used herein, a “3′ overhang” refers to at least one unpaired nucleotide extending from the 3′-end of an RNA strand.

Thus in one embodiment, the siRNA of the invention comprises at least one 3′ overhang of from 1 to about 6 nucleotides (which includes ribonucleotides or deoxynucleotides) in length, preferably from 1 to about 5 nucleotides in length, more preferably from 1 to about 4 nucleotides in length, and particularly preferably from about 2 to about 4 nucleotides in length.

In the embodiment in which both strands of the siRNA molecule comprise a 3′ overhang, the length of the overhangs can be the same or different for each strand. In a most preferred embodiment, the 3′ overhang is present on both strands of the siRNA, and is 2 nucleotides in length. For example, each strand of the siRNA of the invention can comprise 3′ overhangs of dithymidylic acid (“TT”) or diuridylic acid (“uu”).

In order to enhance the stability of the present siRNA, the 3′ overhangs can be also stabilized against degradation. In one embodiment, the overhangs are stabilized by including purine nucleotides, such as adenosine or guanosine nucleotides. Alternatively, substitution of pyrimidine nucleotides by modified analogues, e.g., substitution of uridine nucleotides in the 3′ overhangs with 2′-deoxythymidine, is tolerated and does not affect the efficiency of RNAi degradation. In particular, the absence of a 2′-hydroxyl in the 2′-deoxythymidine significantly enhances the nuclease resistance of the 3′ overhang in tissue culture medium.

In certain embodiments, the siRNA of the invention comprises the sequence AA(N19)TT or NA(N21), where N is any nucleotide. These siRNA comprise approximately 30-70% GC, and preferably comprise approximately 50% G/C. The sequence of the sense siRNA strand corresponds to (N19)TT or N21 (i.e., positions 3 to 23), respectively. In the latter case, the 3′ end of the sense siRNA is converted to TT. The rationale for this sequence conversion is to generate a symmetric duplex with respect to the sequence composition of the sense and antisense strand 3′ overhangs. The antisense RNA strand is then synthesized as the complement to positions 1 to 21 of the sense strand.

Because position 1 of the 23-nucleotide sense strand in these embodiments is not recognized in a sequence-specific manner by the antisense strand, the 3′-most nucleotide residue of the antisense strand can be chosen deliberately. However, the penultimate nucleotide of the antisense strand (complementary to position 2 of the 23-nucleotide sense strand in either embodiment) is generally complementary to the targeted sequence.

In another embodiment, the siRNA of the invention comprises the sequence NAR(N17)YNN, where R is a purine (e.g., A or G) and Y is a pyrimidine (e.g., C or u/T). The respective 21-nucleotide sense and antisense RNA strands of this embodiment therefore generally begin with a purine nucleotide. Such siRNA can be expressed from pol III expression vectors without a change in targeting site, as expression of RNAs from pol III promoters is only believed to be efficient when the first transcribed nucleotide is a purine.

The siRNA of the invention can be targeted to any stretch of approximately 19-25 contiguous nucleotides in any of the target mRNA sequences (the “target sequence”). Techniques for selecting target sequences for siRNA's are given, for example, in Tuschl T et al., “The siRNA User Guide,” revised Oct. 11, 2002, the entire disclosure of which is herein incorporated by reference. “The siRNA User Guide” is available on the world wide web at a website maintained by Dr. Thomas Tuschl, Department of Cellular Biochemistry, AG 105, Max-Planck-Institute for Biophysical Chemistry, 37077 Göttingen, Germany, and can be found by accessing the website of the Max Planck Institute and searching with the keyword “siRNA.” Thus, the sense strand of the present siRNA comprises a nucleotide sequence substantially identical to any contiguous stretch of about 19 to about 25 nucleotides in the target mRNA.

Generally, a target sequence on the target mRNA can be selected from a given cDNA sequence corresponding to the target mRNA, preferably beginning 50 to 100 nt downstream (i.e., in the 3′ direction) from the start codon. The target sequence can, however, be located in the 5′ or 3′ untranslated regions, or in the region nearby the start codon. Suitable target sequences are given in Table 1. Table 1 also shows the dinucleotides AA and TT, which are located, respectively, at the 5′- and 3′-most ends of the target sequences. The target sequences and the target strands in Table 1 comprise the sense strand of the preferred siRNAs of the invention.

TABLE 1
Agnoprotein and Large T Antigen Gene Target Sequences

| Agnoprotein sIRNA Sense Strands | | | | | |
|---|---|---|---|---|---|
| # | nt | | Target Sequence | | Specificity |
| | 48-66 | 5′-AA | AACCTGGAGTGGAACTAAA | dTdT-3′ | JCV |
| | | 5′-AA | AACCTGGACTGGAACAAAA | dTdT-3′ | BKV |
| 1 | 1-19 | 5′-AA | ATGGTTCTTCGCCAGCTGT | dTdT-3′ | JCV |
| 2 | 20-38 | 5′-AA | CACGTAAGGCTTCTGTGAA | dTdT-3′ | JCV |
| 3 | 39-57 | 5′-AA | AGTTAGTAAAACCTGGAGT | dTdT-3′ | JCV |
| 4 | 58-76 | 5′-AA | GGAACTAAAAAAAGAGCTC | dTdT-3′ | JCV |
| 5 | 77-95 | 5′-AA | AAAGGATTTTAATTTTTTTG | dTdT-3′ | JCV |
| 6 | 96-114 | 5′-AA | TTAGAATTTTTGCTGGACT | dTdT-3′ | JCV |
| 7 | 115-133 | 5′-AA | TTTGCACAGGTGAAGACAG | dTdT-3′ | JCV |
| 8 | 134-152 | 5′-AA | TGTAGACGGGAAAAAAAGA | dTdT-3′ | JCV |
| 9 | 153-171 | 5′-AA | CAGAGACACAGTGGTTTGA | dTdT-3′ | JCV |
| 10 | 172-190 | 5′-AA | CTGAGCAGACATACTGTGC | dTdt-3′ | JCV |
| 11 | 191-109 | 5′-AA | CTTGCCTGAACCAAAAGCT | dTdT-3′ | JCV |
| 12 | 10-28 | 5′-AA | CGCCAGCTGTCACGTAAGG | dTdT-3′ | JCV |
| 13 | 30-48 | 5′-AA | TTCTGTGAAAGTTAGTAAA | dTdT-3′ | JCV |
| 14 | 50-68 | 5′-AA | CCTGGAGTGGAACTAAAAA | dTdT-3′ | JCV |
| 15 | 70-98 | 5′-AA | AGAGCTCAAAGGATTTTAA | dTdT-3′ | JCV |
| 16 | 90-118 | 5′-AA | TTTTTTGTTAGAATTTTGC | dTdT-3′ | JCV |
| 17 | 120-138 | 5′-AA | CACAGGTGAAGACAGTGTA | dTdT-3′ | JGV |
| 18 | 130-148 | 5′-AA | GACAGTGTAGACGGGAAAA | dTdT-3′ | JCV, BKV |
| 19 | 160-178 | 5′-AA | CACAGTGGTTTGACTGAGC | dTdT-3′ | JCV |
| 20 | 180-198 | 5′-AA | GACATACAGTGGTTTGCCT | dTdT-3′ | JCV |
| T-antigen siRNA Sense Strands | | | | | |
| # | nt* | | Target Sequence | | Specificity |
| | | 5′-AA | AGTCTTTAGGGTCTTCTACCT | dTdT-3′ | BKV, JCV |
| | | 5′-AA | AGTCCTTGGGGTCTTCTACCT | dTdT-3′ | SV40 |
| 1 | 1-19 | 5′-AA | GTGCCAACCTATGGAACAG | dTdT-3′ | JCV, BKV |
| 2 | 20-38 | 5′-AA | ATGAATGGGAATCCTGGTG | dTdT-3′ | JCV |
| 3 | 60-88 | 5′-AA | GGATGAAGACCTGTTTTGC | dTdT-3′ | JCV |
| 4 | 153-171 | 5′-AA | AAAGGTAGAAGACCCTAAA | dTdT-3′ | JCV, BKV |
| 5 | 170-188 | 5′-AA | AAGACTTTCCTGTAGATCTG | dTdT-3′ | JGV |
| 6 | 210-228 | 5′-AA | TGTGTTTAGTAATAGAACT | dTdT-3′ | JCV, SV4G |
| 7 | 284-302 | 5′-AA | AAACTTATGGAAAAATATT | dTdT-3′ | JCV, BKV |
| 8 | 300-318 | 5′-AA | TTCTGTAACTTTTATAAGT | dTdT-3′ | JCV |
| 9 | 320-338 | 5′-AA | GACATGGTTTTGGGGGTCA | dTdT-3′ | JCV |
| 10 | 340-358 | 5′-AA | AATATTTTGTTTTTCTTAA | dTdT-3′ | JCV |
| 11 | 360-378 | 5′-AA | ACCACATAGACATAGAGTG | dTdT-3′ | JCV |
| 12 | 380-398 | 5′-AA | CAGCAATTAATAACTACTG | dTdT-3′ | JCV |
| 13 | 400-418 | 5′-AA | CAAAAACTATGTACCTTTA | dTdT-3′ | JCV |
| 14 | 420-438 | 5′-AA | TTTTTTAATTTGTAAAGGT | dTdT-3′ | JCV |
| 15 | 440-458 | 5′-AA | TGAATAAGGAATACTTGTT | dTdT-3′ | JCV |
| 16 | 460-478 | 5′-AA | TATAGTGCCCTGTGTAGAC | dTdT-3′ | JCV |
| 17 | 480-498 | 5′-AA | GCCATATGCAGTAGTGGAA | dTdT-3′ | JCV |
| 18 | 500-518 | 5′-AA | AAAGTATTCAGGGGGCCTT | dTdT-3′ | JCV |
| 19 | 520-538 | 5′-AA | AAGGAGCATGACTTTAACC | dTdT-3′ | JCV |
| 20 | 540-558 | 5′-AA | AGAAGAACCAGAAGAAACT | dTdT-3′ | JCV |
| 21 | 560-578 | 5′-AA | AGCAGGTTTCATGGAAATT | dTdT-3′ | JCV |
| 22 | 580-598 | 5′-AA | GTTACACAGTATGCCTTGG | dTdT-3′ | JCV |
| 23 | 600-618 | 5′-AA | AACCAAGTGTGAGGATGTT | dTdT-3′ | JCV |
| 24 | 620-638 | 5′-AA | TTTTGCTTATGGGCATGTA | dTdT-3′ | JCV |
| 25 | 640-658 | 5′-AA | TTAGACTTTCAGGAAAACC | dTdT-3′ | JCV |
| 26 | 660-678 | 5′-AA | ACAGCAATGCAAAAAATGT | dTdT-3′ | JCV |
| 27 | 680-698 | 5′-AA | AAAAAAAGGATCAGCCAAA | dTdT-3′ | JCV |
| 28 | 700-718 | 5′-AA | CACTTTAACCATCATGAAA | dTdT-3′ | JCV |
| 29 | 720-738 | 5′-AA | ACACTATTATAATGCCCAA | dTdT-3′ | JCV |
| 30 | 740-758 | 5′-AA | TTTTTGCAGATAGCAAAAA | dTdT-3′ | JCV |
| 31 | 760-778 | 5′-AA | CAAAAAAGCATTTGCCAGC | dTdT-3′ | JCV |
| 32 | 780-798 | 5′-AA | GGCTGTTGATACTGTAGCA | dTdT-3′ | JCV |
| 33 | 800-818 | 5′-AA | CCAAACAAAGGGTTGACAG | dTdT-3′ | JCV |
| 34 | 820-838 | 5′-AA | ATCCACATGACCAGAGAAG | dTdT-3′ | JCV |
| 35 | 840-858 | 5′-AA | AATGTTAGTTGAAAGGTTT | dTdT-3′ | JCV |
| 36 | 860-878 | 5′-AA | ATTTCTTGCTTGATAAAAT | dTdT-3′ | JCV |
| 37 | 880-898 | 5′-AA | GACTTAATTTTTGGGGCAC | dTdT-3′ | JCV |
| 38 | 900-918 | 5′-AA | TGGCAATGCTGTTTTAGAG | dTdT-3′ | JCV |
| 39 | 920-938 | 5′-AA | AATATATGGCTGGGGTGGC | dTdT-3′ | JCV |
| 40 | 940-958 | 5′-AA | TGGATTCATTGCTTGCTGC | dTdT-3′ | JCV |
| 41 | 960-978 | 5′-AA | TCAAATGGACACTGTTATT | dTdT-3′ | JCV |
| 42 | 980-998 | 5′-AA | ATGACTTTCTAAAATGCAT | dTdT-3′ | JCV |
| 43 | 1000-1018 | 5′-AA | GTATTAAACATTCCAAAAA | dTdT-3′ | JCV |
| 44 | 1020-1038 | 5′-AA | AAGGTACTGGGTATTCAGG | dTdT-3′ | JCV |
| 45 | 1040-1058 | 5′-AA | GGCCAATAGACAGTGGCAA | dTdT-3′ | JCV |
| 46 | 1060-1078 | 5′-AA | AGTAGTTTAGCTGCAGGTT | dTdT-3′ | JCV |
| 47 | 1080-1098 | 5′-AA | AGTTGATCTGTGTGGGGGA | dTdT-3′ | Joy |
| 48 | 1100-1118 | 5′-AA | ACTGATTAAATGTTAATAT | dTdT-3′ | JCV |
| 49 | 1120-1138 | 5′-AA | GGCATTAGAAAGATTAAAC | dTdT-3′ | JCV |
| 50 | 1140-1158 | 5′-AA | TGAATTAGGAGTGGGTATA | dTdT-3′ | JCV |

*numbered from exon of T-antigen, which begins at nt 247.

It is understood that the target sequences given in Table 1 are with reference to the agnoprotein gene or large T antigen gene cDNA, and thus these sequences contain deoxythymidines represented by “T.” One skilled in the art would understand that, in the actual target sequence of the mRNA, the deoxythymidines would be replaced by uridines (“u”). Likewise, a target sequence contained within an siRNA of the invention would also contain uridines in place of deoxythymidines, except where the siRNA comprise dithymidylic acid 3′-overhangs.

The siRNA of the invention can be obtained using a number of techniques known to those of skill in the art. For example, the siRNA can be chemically synthesized or recombinantly produced using methods known in the art, such as the Drosophila in vitro system described in U.S. published application 2002/0086356 of Tuschl et al., the entire disclosure of which is herein incorporated by reference.

Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK).

Alternatively, siRNA can also be expressed from recombinant circular or linear DNA plasmids using any suitable promoter. Suitable promoters for expressing siRNA of the invention from a plasmid include, for example, the U6 or H1 RNA pol III promoter sequences and the cytomegalovirus promoter. Selection of other suitable promoters is within the skill in the art. The recombinant plasmids of the invention can also comprise inducible or regulatable promoters for expression of the siRNA in a particular tissue or in a particular intracellular environment.

The siRNA expressed from recombinant plasmids can either be isolated from cultured cell expression systems by standard techniques, or can be expressed intracellularly. The use of recombinant plasmids to deliver siRNA of the invention to cells in vivo is discussed in more detail below.

siRNA of the invention can be expressed from a recombinant plasmid either as two separate, complementary RNA molecules, or as a single RNA molecule with two complementary regions.

Selection of plasmids suitable for expressing siRNA of the invention, methods for inserting nucleic acid sequences for expressing the siRNA into the plasmid, and methods of delivering the recombinant plasmid to the cells of interest are within the skill in the art. See, for example Tuschl, T. (2002), Nat. Biotechnol, 20: 446-448; Brummelkamp T R et al. (2002), Science 296: 550-553; Miyagishi M et al. (2002), Nat. Biotechnol. 20: 497-500; Paddison P J et al. (2002), Genes Dev. 16: 948-958; Lee N S et al. (2002), Nat. Biotechnol. 20: 500-505; and Paul C P et al. (2002), Nat. Biotechnol. 20: 505-508, the entire disclosures of which are herein incorporated by reference.

In one embodiment, a plasmid expressing an siRNA of the invention comprises a sense RNA strand coding sequence in operable connection with a polyT termination sequence under the control of a human U6 RNA promoter, and an antisense RNA strand coding sequence in operable connection with a polyT termination sequence under the control of a human U6 RNA promoter. Such a plasmid can be used in producing an recombinant adeno-associated viral vector for expressing an siRNA of the invention.

As used herein, “in operable connection with a polyT termination sequence” means that the nucleic acid sequences encoding the sense or antisense strands are immediately adjacent to the polyT termination signal in the 5′ direction. During transcription of the sense or antisense sequences from the plasmid, the polyT termination signals act to terminate transcription.

As used herein, “under the control” of a promoter means that the nucleic acid sequences encoding the sense or antisense strands are located 3′ of the promoter, so that the promoter can initiate transcription of the sense or antisense coding sequences.

The siRNA of the invention can also be expressed from recombinant viral vectors intracellularly in vivo. The recombinant viral vectors of the invention comprise sequences encoding the siRNA of the invention and any suitable promoter for expressing the siRNA sequences. Suitable promoters include, for example, the U6 or H1 RNA pol III promoter sequences and the cytomegalovirus promoter. Selection of other suitable promoters is within the skill in the art. The recombinant viral vectors of the invention can also comprise inducible or regulatable promoters for expression of the siRNA in a particular tissue or in a particular intracellular environment. The use of recombinant viral vectors to deliver siRNA of the invention to cells in vivo is discussed in more detail below.

siRNA of the invention can be expressed from a recombinant viral vector either as two separate, complementary RNA molecules, or as a single RNA molecule with two complementary regions.

Any viral vector capable of accepting the coding sequences for the siRNA molecule(s) to be expressed can be used, for example vectors derived from adenovirus (AV); adeno-associated virus (AAV); retroviruses (e.g, lentiviruses (LV), Rhabdoviruses, murine leukemia virus); herpes virus, and the like. The tropism of viral vectors can be modified by pseudotyping the vectors with envelope proteins or other surface antigens from other viruses, or by substituting different viral capsid proteins, as appropriate.

For example, lentiviral vectors of the invention can be pseudotyped with surface proteins from vesicular stomatitis virus (VSV), rabies, Ebola, Mokola, and the like. AAV vectors of the invention can be made to target different cells by engineering the vectors to express different capsid protein serotypes. For example, an AAV vector expressing a serotype 2 capsid on a serotype 2 genome is called AAV 2/2. This serotype 2 capsid gene in the AAV 2/2 vector can be replaced by a serotype 5 capsid gene to produce an AAV 2/5 vector. Techniques for constructing AAV vectors which express different capsid protein serotypes are within the skill in the art; see, e.g., Rabinowitz J E et al. (2002), J Virol 76:791-801, the entire disclosure of which is herein incorporated by reference.

Selection of recombinant viral vectors suitable for use in the invention, methods for inserting nucleic acid sequences for expressing the siRNA into the vector, and methods of delivering the viral vector to the cells of interest are within the skill in the art. See, for example, Dornburg R (1995), Gene Therap. 2: 301-310; Eglitis MA (1988), Biotechniques 6: 608-614; Miller AD (1990), Hum Gene Therap. 1: 5-14; Anderson WF (1998), Nature 392: 25-30; and Rubinson D A et al., Nat. Genet. 33: 401-406, the entire disclosures of which are herein incorporated by reference.

Preferred viral vectors are those derived from AV and AAV. In a particularly preferred embodiment, the siRNA of the invention is expressed as two separate, complementary single-stranded RNA molecules from a recombinant AAV vector comprising, for example, either the U6 or H1 RNA promoters, or the cytomegalovirus (CMV) promoter.

A suitable AV vector for expressing the siRNA of the invention, a method for constructing the recombinant AV vector, and a method for delivering the vector into target cells, are described in Xia H et al. (2002), Nat. Biotech. 20: 1006-1010.

Suitable AAV vectors for expressing the siRNA of the invention, methods for constructing the recombinant AV vector, and methods for delivering the vectors into target cells are described in Samulski R et al. (1987), J. Virol. 61: 3096-3101; Fisher K J et al. (1996), J. Virol., 70: 520-532; Samulski R et al. (1989), J. Virol. 63: 3822-3826; U.S. Pat. No. 5,252,479; U.S. Pat. No. 5,139,941; International Patent Application No. WO 94/13788; and International Patent Application No. WO 93/24641, the entire disclosures of which are herein incorporated by reference.

The ability of an siRNA containing a given target sequence to cause RNAi-mediated degradation of the target mRNA can be evaluated using standard techniques for measuring the levels of RNA or protein in cells. For example, siRNA of the invention can be delivered to cultured cells infected with JCV, and the levels of target mRNA can be measured by Northern blot or dot blotting techniques, or by quantitative RT-PCR. Alternatively, the levels of agnoprotein or large T antigen in the cultured cells can be measured by ELISA or Western blot. Suitable protocols for infecting cultured cells with JCV, the delivery of siRNA to cultured cells, and assays for detecting protein and mRNA levels in cultured cells, are within the skill in the art (see, e.g., the Examples below).

As discussed above, the siRNA of the invention target can cause the RNAi-mediated degradation of JCV agnoprotein gene or large T antigen gene mRNA, or alternative splice forms or mutants thereof. If the siRNA of the invention comprises a target sequence in common with other primate polyomavirus genes (e.g., genes from BKV and/or SV40), the siRNA can cause the degradation of mRNA produced from these other primate polyomavirus genes as well. Degradation of the target mRNA by the present siRNA reduces the production of a functional gene product from these genes, and also prevents or reduces expression of other JCV genes (such as the VP1 gene). Thus, the invention provides a method of inhibiting expression of agnoprotein gene, large T antigen gene, or other JCV genes (such as VP1) in a subject, comprising administering an effective amount of an siRNA of the invention to the subject, such that the target mRNA is degraded. The invention also provides a method of inhibiting JCV virus infection and/or replication in a subject by the RNAi-mediated degradation of the target mRNA by the present siRNA.

The siRNA of the invention can also be used to treat diseases associated with JCV or other primate polyomavirus infection, such as are known in the art. For example, JCV infection is associated with cancer of central nervous system (CNS) origin (including medullablastoma and glioblastoma) the demyelinating disease PML. Thus, the invention provides a method of treating diseases associated with JCV, BKV and/or SV40 infection, comprising administering to a subject an effective amount of one or more siRNA of the invention.

As used herein, a “disease associated with JCV infection” means any disease or disorder which is correlated with the presence of an active or latent JCV infection. Exemplary diseases associated with JCV infection are CNS cancers such as medullablastoma and glioblastoma, and demyelinating diseases such as PML. As used herein, a “disease associated with BKV infection” means any disease or disorder which is correlated with the presence of an active or latent BKV infection, and includes polyomavirus-associated neuropathy. As used herein, a “disease associated with SV40 infection” means any disease or disorder which is correlated with the presence of an active or latent SV40 infection, and includes SV40-induced cancers such as mesothelioma.

As used herein, to “treat” a disease associated with JCV infection means that symptoms associated with the disease are do not worsen, are reduced or are prevented. Evaluation of symptoms of diseases associated with JCV infection are within the skill in the art.

In the practice of the present methods, two or more siRNA comprising different target sequences in the JCV agnoprotein gene or large T antigen gene mRNA can be administered to the subject. In a preferred embodiment, two or more siRNA, each comprising target sequences from JCV agnoprotein gene and large T antigen gene mRNA, are be administered to a subject.

As used herein, a “subject” includes a human being or non-human animal. Preferably, the subject is a human being.

As used herein, an “effective amount” of the siRNA is an amount sufficient to cause RNAi-mediated degradation of the target mRNA, or an amount sufficient to treat a disease associated with JCV, BKV and/or SV40 infection in a subject.

RNAi-mediated degradation of the target mRNA can be detected by measuring levels of the target mRNA or protein in the cells of a subject, using standard techniques for isolating and quantifying mRNA or protein as described above.

It is understood that the siRNA of the invention can mediate RNA interference in substoichiometric amounts. Without wishing to be bound by any theory, it is believed that the siRNA of the invention induces the RISC to degrade the target mRNA in a catalytic manner. Thus, compared to standard techniques for inhibiting primate polyomavirus (e.g., JCV, BKV and/or SV40) replication or infection, significantly less siRNA needs to be administered to the subject to have a therapeutic effect.

One skilled in the art can readily determine an effective amount of the siRNA of the invention to be administered to a given subject, by taking into account factors such as the size and weight of the subject; the extent of the JCV infection or disease penetration; the age, health and sex of the subject; the route of administration; and whether the administration is regional or systemic. Generally, an effective amount of the siRNA of the invention comprises an intercellular concentration at or near the neovascularization site of from about 1 nanomolar (nM) to about 100 nM, preferably from about 2 nM to about 50 nM, more preferably from about 2.5 nM to about 10 nM. Particularly preferred effective amounts of the siRNA of the invention can comprise an intercellular concentration at or near the neovascularization site of about 1 nM, about 5 nM, or about 25 nM. It is contemplated that greater or lesser effective amounts of siRNA can be administered.

For treating diseases associated with JCV, BKV and/or SV40 infection, the siRNA of the invention can administered to a subject in combination with a pharmaceutical agent which is different from the present siRNA. For example, the siRNA of the invention can be administered in combination with therapeutic methods currently employed for treating cancer or preventing tumor metastasis (e.g., radiation therapy, chemotherapy, and surgery). For treating tumors, the siRNA of the invention is preferably administered to a subject in combination with radiation therapy, or in combination with chemotherapeutic agents such as cisplatin, carboplatin, cyclophosphamide, 5-fluorouracil, adriamycin, daunorubicin or tamoxifen.

In the present methods, the siRNA can be administered to the subject either as naked siRNA, in conjunction with a delivery reagent, or as a recombinant plasmid or viral vector which expresses the siRNA.

Suitable delivery reagents for administration in conjunction with the present siRNA include the Mirus Transit TKO lipophilic reagent; lipofectin; lipofectamine; cellfectin; or polycations (e.g., polylysine), or liposomes. A preferred delivery reagent is a liposome.

Liposomes can aid in the delivery of the siRNA to a particular tissue, such as retinal or tumor tissue, and can also increase the blood half-life of the siRNA. Liposomes suitable for use in the invention are formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al. (1980), Ann. Rev. Biophys. Bioeng. 9: 467; and U.S. Pat. Nos. 4,235,871, 4,501,728, 4,837,028, and 5,019,369, the entire disclosures of which are herein incorporated by reference.

Preferably, liposomes encapsulating the present siRNA comprise a ligand molecule that can target the liposome to cells which are infected with primate polyomavirus; e.g., JCV, BKV and/or SV40.

Particularly preferably, the liposomes encapsulating the present siRNA are modified so as to avoid clearance by the mononuclear macrophage and reticuloendothelial systems, for example by having opsonization-inhibition moieties bound to the surface of the structure. In one embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, tissue characterized by such microvasculature defects, for example solid tumors, will efficiently accumulate these liposomes; see Gabizon, et al. (1988), P.N.A.S., USA, 18: 6949-53. In addition, the reduced uptake by the RES lowers the toxicity of stealth liposomes by preventing significant accumulation in the liver and spleen. Thus, liposomes of the invention that are modified with opsonization-inhibition moieties are particularly suited to deliver the present siRNA to tumor cells.

Opsonization inhibiting moieties suitable for modifying liposomes are preferably water-soluble polymers with a number average molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM₁. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

Preferably, the opsonization-inhibiting moiety is a PEG, PPG, or derivatives thereof. Liposomes modified with PEG or PEG-derivatives are sometimes called “PEGylated liposomes.”

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture such as tetrahydrofuran and water in a 30:12 ratio at 60° C.

Recombinant plasmids which express siRNA of the invention are discussed above. Such recombinant plasmids can also be administered directly or in conjunction with a suitable delivery reagent, including the Mirus Transit LT1 lipophilic reagent; lipofectin; lipofectamine; cellfectin; polycations (e.g., polylysine) or liposomes. Recombinant viral vectors which express siRNA of the invention are also discussed above, and methods for delivering such vectors to cells of a subject which are infected with JCV are within the skill in the art.

The siRNA of the invention can be administered to the subject by any means suitable for delivering the siRNA to cells infected with primate polyomavirus; e.g., JCV, BKV and/or SV40. For example, the siRNA can be administered by gene gun, electroporation, or by other suitable parenteral or enteral administration routes.

Suitable enteral administration routes include oral, rectal, or intranasal delivery.

Suitable parenteral administration routes include intravascular administration (e.g. intravenous bolus injection, intravenous infusion, intra-arterial bolus injection, intra-arterial infusion and catheter instillation into the vasculature); peri- and intra-tissue administration (e.g., peri-tumoral and intra-tumoral injection, intra-retinal injection or subretinal injection); subcutaneous injection or deposition including subcutaneous infusion (such as by osmotic pumps); direct (e.g., topical) application to the area at or near the site of neovascularization, for example by a catheter or other placement device (e.g., a corneal pellet or a suppository, eye-dropper, or an implant comprising a porous, non-porous, or gelatinous material); and inhalation. Suitable placement devices include the ocular implants described in U.S. Pat. Nos. 5,902,598 and 6,375,972, and the biodegradable ocular implants described in U.S. Pat. No. 6,331,313, the entire disclosures of which are herein incorporated by reference. Such ocular implants are available from Control Delivery Systems, Inc. (Watertown, Mass.) and Oculex Pharmaceuticals, Inc. (Sunnyvale, Calif.).

In a preferred embodiment, injections or infusions of the siRNA are given at or near the site of primate polyomavirus infection.

The siRNA of the invention can be administered in a single dose or in multiple doses. Where the administration of the siRNA of the invention is by infusion, the infusion can be a single sustained dose or can be delivered by multiple infusions.

One skilled in the art can also readily determine an appropriate dosage regimen for administering the siRNA of the invention to a given subject. For example, the siRNA can be administered to the subject once, such as by a single injection or deposition at or near the site of primate polyomavirus infection. Alternatively, the siRNA can be administered to a subject multiple times daily or weekly. For example, the siRNA can be administered to a subject once weekly for a period of from about three to about twenty-eight weeks, more preferably from about seven to about ten weeks. In a preferred dosage regimen, the siRNA is injected at or near the site of primate polyomavirus infection once a week for seven weeks. It is understood that periodic administrations of the siRNA of the invention for an indefinite length of time may be necessary for subjects suffering from a demyelinating disease such as PML.

Where a dosage regimen comprises multiple administrations or the administration of two or more siRNA, each of which comprise a different target sequence, it is understood that the effective amount of siRNA administered to the subject can comprise the total amount of siRNA administered over the entire dosage regimen.

The siRNA of the invention are preferably formulated as pharmaceutical compositions prior to administering to a subject, according to techniques known in the art. Pharmaceutical compositions of the present invention are characterized as being at least sterile and pyrogen-free. As used herein, “pharmaceutical formulations” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is herein incorporated by reference.

The present pharmaceutical formulations comprise an siRNA of the invention (e.g., 0.1 to 90% by weight), or a physiologically acceptable salt thereof, mixed with a physiologically acceptable carrier medium. Preferred physiologically acceptable carrier media are water, buffered water, normal saline, 0.4% saline, 0.3% glycine, hyaluronic acid and the like.

Pharmaceutical compositions of the invention can also comprise conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can comprise any of the carriers and excipients listed above and 10-95%, preferably 25%-75%, of one or more siRNA of the invention. A pharmaceutical composition for aerosol (inhalational) administration can comprise 0.01-20% by weight, preferably 1%-10% by weight, of one or more siRNA of the invention encapsulated in a liposome as described above, and propellant. A carrier can also be included as desired; e.g., lecithin for intranasal delivery.

The invention will now be illustrated by the following non-limiting examples.

### EXAMPLES

As the effective inhibition of JCV gene expression and replication is the first and most critical step in the treatment of PML, we have utilized RNA interference for targeting expression of the viral regulatory proteins expressed by the early (T-antigen) and late (Agnoprotein) genome. Our results have shown that combined treatment of the infected cells with siRNA targeting T-antigen and Agnoprotein completely abrogated production of the viral capsid proteins in glial cells.

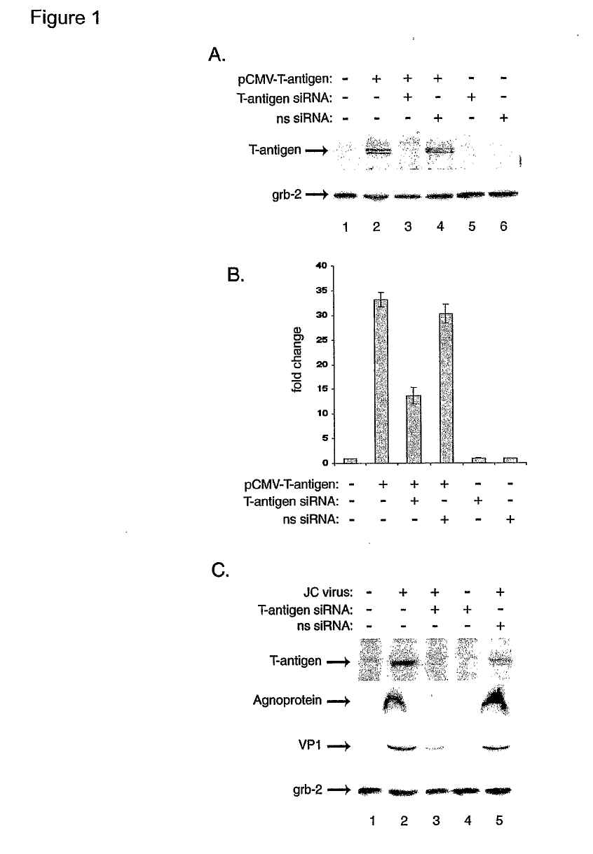

In the first series of experiments, we assessed the ability of our designed siRNA to suppress expression of JCV T-antigen. Human primary fetal astrocytes were transfected with a plasmid expressing T-antigen (pCMV-T-antigen) and cells were subsequently transfected with the siRNA oligonucleotides targeting T-antigen. As shown in FIG. 1A, treatment of cells with T-antigen siRNA decreased the level of T-antigen, but not production of the unrelated cellular protein, Grb-2. To further demonstrate the suppression of T-antigen by siRNA, we performed a functional assay in which the level of the JCV late promoter (JCVL) activation by T-antigen was tested in astrocytes upon treatment of cells with T-antigen siRNA. Results showed more than a 50% decrease in the level of JCVL transcriptional activation by T-antigen, indicating that the observed reduction in the level of T-antigen by siRNA has a functional consequence on its ability to stimulate expression of the viral late genome. To examine the ability of T-antigen siRNA to suppress viral gene expression during the course of infection, primary human fetal astrocytes were infected with the Mad-4 strain of JCV and at days 1, 5, and 10 post-infection, cells were transfected with T-antigen siRNA oligonucleotides. Results from analysis of viral proteins at 15 days post-infection showed drastic suppression of T-antigen, a marginal decrease in Agnoprotein, and a noticeable reduction in the level of the viral capsid protein, VP1, in the siRNA-treated cells in comparison with control cells. Neither viral infection nor siRNA treatment influenced the level of grb-2 production.

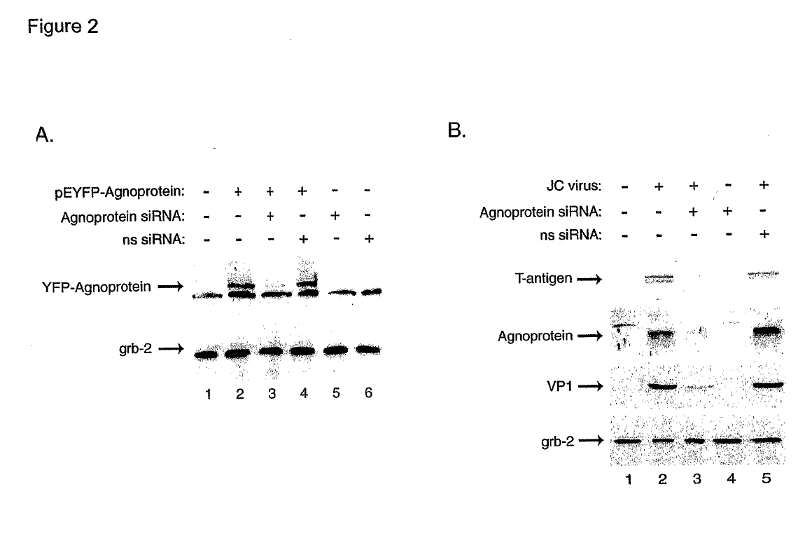

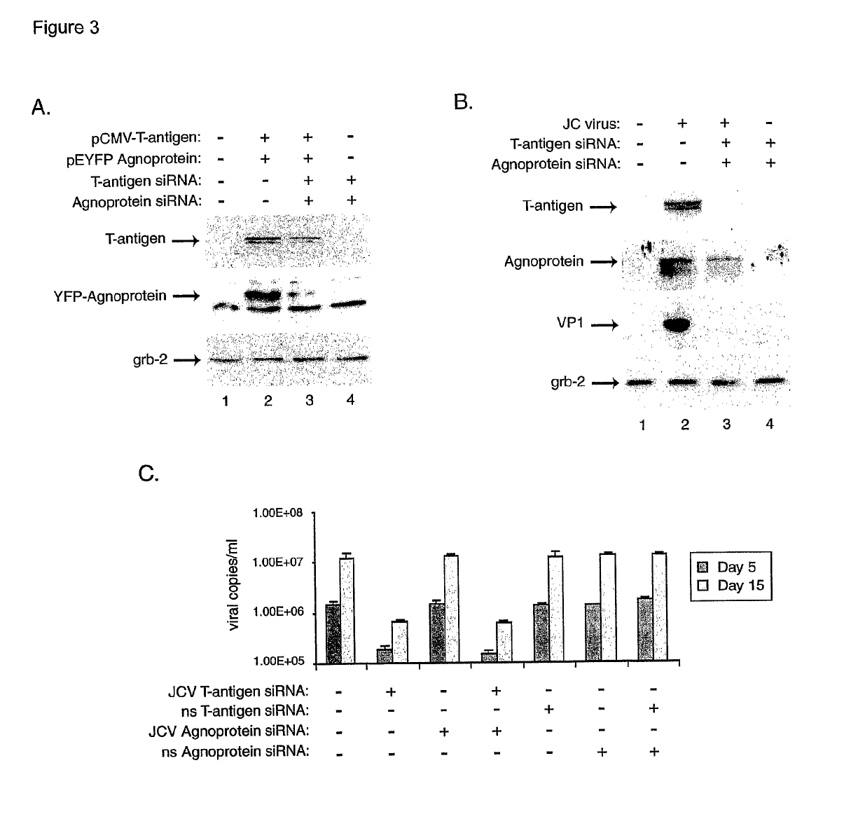

As our strategy for targeting T-antigen by siRNA had only a partial effect on the production of Agnoprotein and the major capsid protein, VP1, in a second approach we designed and employed siRNA targeting expression of the JCV Agnoprotein. As before, the efficacy of Agnoprotein siRNA in silencing expression of Agnoprotein was first tested by transfection assay. As seen in FIG. 2A, transfection of cells expressing YFP-Agnoprotein with Agnoprotein siRNA oligonucleotides drastically suppressed production of Agnoprotein in the cells. Interestingly, siRNA designed for Agnoprotein from the polyomavirus, BKV, which shares a two nucleotide mismatch with the analogous region of the JCV genome failed to suppress expression of JCV Agnoprotein in the transfected cells, verifying the high level of specificity of the designed siRNAs. Similarly, JCV Agnoprotein siRNA, but not BKV Agnoprotein siRNA drastically suppressed the production of JCV Agnoprotein in JCV infected human fetal astrocytes. Of particular interest was the notion that the extinction of Agnoprotein by siRNA affected production of the viral early protein, T-antigen. This observation suggests that Agnoprotein promotes the expression of T-antigen. Also, a noticeable decrease in the level of VP 1 was detected in cells treated with JCV Agnoprotein siRNA.

In the next set of experiments we utilized both T-antigen and Agnoprotein siRNAs in combination to block expression of the viral early and late regulatory proteins and to test the expression level of the viral major capsid protein. Results from co-transfection of cells with plasmids expressing T-antigen and YFP-Agnoprotein showed that co-treatment of cells with both siRNAs drastically reduced the levels of both T-antigen and Agnoprotein in the cells. In JCV-infected cells, co-treatment with siRNAs completely abrogated expression of T-antigen and VP1, as no bands corresponding to either protein were detected by Western blot analysis of the protein extracts from the infected and treated cells. The level of Agnoprotein was decreased in the infected cells upon co-treatment of cells with both siRNAs. Complete suppression of T-antigen and VP1 by T-antigen and Agnoprotein siRNAs indicated the effectiveness of the combined treatment in blocking viral gene expression and protein products.

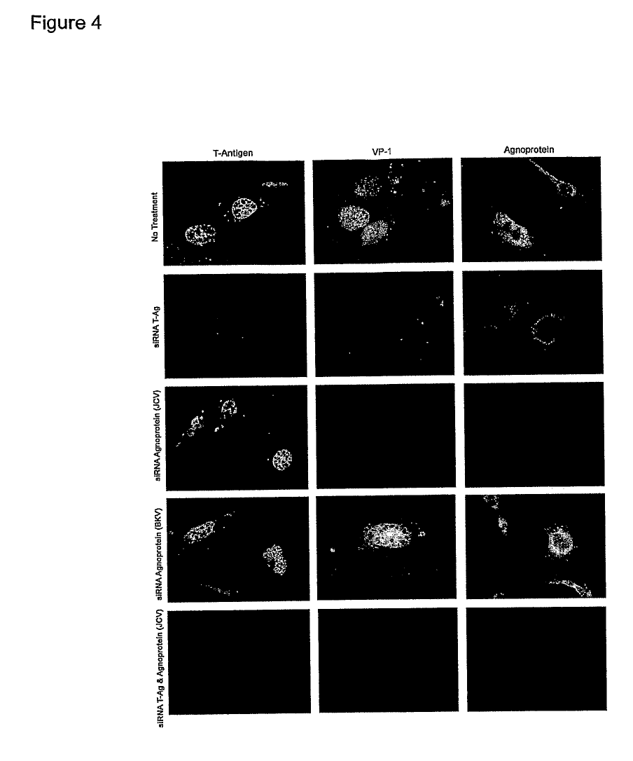

In an alternative approach, we employed immunocytochemistry to assess the level and subcellular location of viral proteins in the infected cells upon treatment with siRNAs targeting one or both viral regulatory proteins. As shown in FIG. 4, a high level of T-antigen and VP1 were expressed and were appropriately localized in the nuclei of JCV infected primary human fetal astrocytic cells. Also, in accord with previous reports (Del Valle et al, 2001b; Radhakrishnan et al, 2003; Okada et al, 2001), high levels of Agnoprotein with cytoplasmic perinuclear accumulation were observed in the JCV-infected cells. Treatment of the cells with T-antigen siRNA caused a drastic decrease in the expression of both T-antigen and VP I, but had a marginal effect on Agnoprotein production. Treatment of cells with Agnoprotein siRNA resulted in a major decline in the levels of Agnoprotein and VP1 and resulted in suppression of T-antigen appearance in the nuclei of some, but not all, infected cells. The observed events were specific, as under similar conditions, BKV Agnoprotein siRNA had no effect on the production of T-antigen, VP1, or Agnoprotein. Co-treatment of infected cells with siRNAs for targeting JCV T-antigen and Agnoprotein resulted in silencing of both viral early and late gene expression indicating that co-treatment of the infected cells with siRNAs for the two viral regulatory proteins can effectively block viral gene expression in JCV-infected human astrocytes.

Intracellular immunization against virus replication in mammalian cells can be accomplished by expression of proteins and/or RNAs that effectively interfere with viral gene expression. In this respect, the use of RNA interference which prevents the expression of genes by using small molecules, such as small interfering RNAs and siRNAs, has recently received special attention due to their ability to effect viral gene expression (Gitlin et al, 2002; Jacque et al, 2002; Ying et al, 2003; Chang and Taylor, 2003). Once formed, siRNAs can be incorporated into a protein complex that recognizes and cleaves target mRNAs (Gitlin and Andino, 2003). In this study, we directed a 21-nucleotide siRNA duplex against two regions of the JCV genome that correspond to the viral early regulatory protein, T-antigen, and the viral late regulatory protein, Agnoprotein. Our rationale for targeting the early genome of JCV is based on established information indicating that expression of T-antigen is an essential early event in initiating the viral lytic cycle that includes viral DNA replication and expression of the viral late genome responsible for capsid proteins. Our results show that while one siRNA duplex for T-antigen can decrease the level of T-antigen, there exists sufficient T-antigen that stimulates, albeit at a decreased level, i.e. greater than 50%, late gene expression and capsid production. While T-antigen siRNA exhibited a more robust effect in infected cells, it should be noted that in the transient transfection assay, siRNA was transfected 24 h after the T-antigen plasmid and therefore may not have been delivered into the identical cells receiving the T-antigen expression vector. Furthermore, studies in stable cell lines show this siRNA completely abrogates T-antigen expression (data not shown).

In light of previous results ascribing a regulatory function for Agnoprotein, we developed siRNA for targeting Agnoprotein production and showed that its use can also decrease the level of viral early and late protein production by more than 50%. We have also observed the ability of Agnoprotein siRNA to significantly down-regulate T-antigen expression has at both the RNA and protein level (Safak et al, unpublished observations). Once combined, we were able to completely abolish production of the JCV capsid protein, VP I, though neither siRNA directly targeted this protein. In light of the fact that VP1, the major capsid protein, is essential for production of infectious virions, it is quite interesting to note that VP1 expression can be blocked via targeting of T-antigen and Agnoprotein, thus abrogating productive infection. These data indicate that expression of the JCV genome can be effectively controlled by RNA interference technology. The genomic stability of double-stranded DNA viruses such as JCV and the fact that both T-antigen and Agnoprotein are essential for viral replication makes these two proteins attractive therapeutic targets.

### REFERENCES

Aksamit, A. 2001. Treatment of non-AIDS progressive multifocal leukoencephalopathy with cytosine arabinoside. J. Neurovirol. 7:386-390.
Berger, J. R. and M. Concha. 1995. Progressive multifocal leukoencephalopathy: the evolution of a disease once considered rare. J. Neurovirol. 1:5-18.
Berger, J. R., B. Kaszovitz, J. D. Post, and G. Dickinson. 1987. Progressive multifocal leukoencephalopathy associated with human immunodeficiency virus infection: a review of the literature with a report of sixteen cases. Ann. Int. Med. 107:78-87.
Chang, J., and J. M. Taylor. 2003. Susceptibility of human hepatitis delta virus RNAs to small interfering RNA action. J. Virol. 77:9728-31.
Cinque, P., Koralnik, I. J., Clifford, D. B. 2003. The evolving face of human immunodeficiency virus-related progressive multifocal leukoencephalopathy: Defining a consensus terminology. J. Neurovirol. 9(S 1):88-92.
Clifford, D. B, and E. O. Major. 2001. The biology of JC virus and progressive multifocal leukoencephalopathy. J. Neurovirol. 4:279.
Darbinyan, A., N. Darbinian, M. Safak, S. Radhakrishnan, A. Giordano, and K. Khalili. 2002. Evidence for dysregulation of cell cycle by human polyomavirus, JCV, late auxiliary protein. Oncogene 21:5574-5581.
Del Valle, L., J. Gordon, S. Enam, S. Delbue, S. Croul., S. Abraham, S. Radhakrishnan, M. Assimakoupoulou, C. D. Katsetos, and K. Khalili. 2001a. Expression of human neurotropic polyomavirus JCV late gene product AGNO proteiri in human medulloblastoma. J. Natl. Cancer Inst 94:267-273.
Del Valle, L., J. Gordon, M. Assimakopoulou, S. Enam, J. F. geddes, J. N. Varakis, C. D. Katsetos, S. Croul, and K. Khalili. 2001b. Detection of JC virus DNA sequences and expression of the viral regulatory protein T-antigen in tumors of the central nervous system. Cancer Res. 61:428793
Frisque, R. J. and R. A. White III. 1992. The molecular biology of JCV, causative agent of progressive multifocal leukoencephalopathy. In: Molecular neurovirology, R. P. Roos (ed). Humana Press, Totowa, N.J., pp. 25-158.
Gitlin, L., and R. Andino. 2003. Nucleic acid-based immune system: the antiviral potential of mammalian RNA silencing. J. Virol. 77:7159-65.
Gitlin, L., S. Karelsky, and R. Andino. 2002. Short interfering RNA confers intracellular antiviral immunity in human cells. Nature 418:430-34
Gordon, J., and K. Khalili. 1998. The human polyomavirus, JCV, and neurologic diseases. Int. J. Mol. Med. 1:647-55.
Hall, C. D., U. Dafni, D. Simpson, D., Clifford, P. E. Wetherill, B. Cohen, J. McArthur, H. Hollander, C. Yainnoutsos, E. Major, L. Millar, and J. Timpone. 1998. Failure of cytarabine in progressive multifocal leukoencephalopathy associated with human immunodeficiency virus infection. AIDS Clinical Trials Group 243 Team. N. Engl. J. Med. 338:1345-51.
Jacque, J.-M., K. Triques, and M. Stevenson. 2002. Modulation of HIV-1 replication by RNA interference. Nature 418:435-8
Kerr, D. A., C. -F. Chang, J. Gordon, M. -A. Bjornsti, and K. Khalili. 1993. Inhibition of human neurotropic virus (JCV) DNA replication in glial cells by camptothecin. Virology 196:612-18.
Khalili, K., L, Del Valle, J. Otte, M. Weaver, and J. Gordon. 2003. Human neurotropic polyomavirus, JCV, and its role in carcinogenesis. Oncogene 22:5181-5191.
Levy, R. M., E. Major, M. J. Ali, B. Cohen, and D. Groothius. 2001. Convection-enhanced intraparenchymal delivery (CEID) of cytosine arabinoside (AraC) for the treatment of HIV-related progressive multifocal leukoencephalopathy (PML). J. Neurovirol 7:382-85.
Okada, Y., S. Endo, H. Takahashi, H. Sawa, T. Umemura, and K. Nagashima. 2001. Distribution and function of JCV agnoprotein. J. Neurovirol. 7:302-306.
Radhakrishnan, S., J. Otte, S. Enam, L. Del Valle, K. Khalili, and J. Gordon. 2003. JC virus-induced changes in cellular gene expression in primary human astrocytes. J. Virol. 77:10638-44.
Raj, G. V., and K. Khalili. 1995. Transcriptional regulation: Lessons from the human neurotropic polyomavirus, JCV. Virology 10:283-91.
Royal, W. III, B. Dupont, D. mcGuire, L Chang, K. Gookin, T. Ernst, M. J. Post, D. Fish, G. Pailloux, H. Poncelet, M. Concha, L. Appuzzo, and E. Singer. 2003. Topotecan in the treatment of acquired immunodeficiency syndrome-related progressive multifocal leukoecephalopathy. J. Neurovirol. 9:411-419.
Sweeney, B. J., Manji, H., Miller, R. F., harrison, M. J., Gray, F., and Scaravilli, F. 1994. Cortical and subcortical JC virus infection: two unusual cases of AIDS associated progressive multifocal leukoencephalopathy. J. Neurol. Neurosurg. Psychiatry 57:994-997.
Walker, D. L. and B. L. Padgett 1983. The epidemiology of human papovaviruses, in Polyomaviruses and Human Neurological Disease. Sever, J. L. and D. L. Madden, eds., Alan R. Liss, New York, pp. 99-106.
Ying, C., E. De Clercq, and J. Neyts. 2003. Selective inhibition of hepatitis B virus replication by RNA interference. Biochem. Biophys. Res. Comm. 309:482-84.

All documents referred to herein are incorporated by reference. While the present invention has been described in connection with the preferred embodiments and the various figures, it is to be understood that other similar embodiments may be used or modifications and additions made to the described embodiments for performing the same function of the present invention without deviating therefrom. Therefore, the present invention should not be limited to any single embodiment, but rather should be construed in breadth and scope in accordance with the recitation of the appended claims.

1-63. (canceled)

64. An isolated siRNA comprising a sense RNA strand and an antisense RNA strand, wherein the sense and antisense RNA strands form an RNA duplex, and wherein the sense RNA strand comprises a nucleotide sequence substantially identical to a target sequence of about 19 to about 25 contiguous nucleotides in agnoprotein gene or large T antigen gene mRNA of primate polyomviruses, or an alternative splice form or mutant thereof.

65. The siRNA of claim 64, wherein the primate polyomavirus agnoprotein gene or large T antigen gene target sequence is also contained in an mRNA produced from a JCV, BKV or SV40 gene.

66. The siRNA of claim 64, wherein the sense and antisense RNA strands forming the RNA duplex are covalently linked by a single-stranded hairpin.

67. A recombinant plasmid or recombinant viral vector comprising nucleic acid sequences for expressing an siRNA comprising a sense RNA strand and an antisense RNA strand, wherein the sense and an antisense RNA strands form an RNA duplex, and wherein the sense RNA strand comprises a nucleotide sequence substantially identical to a target sequence of about 19 to about 25 contiguous nucleotides in agnoprotein gene or large T antigen gene mRNA of primate polyomaviruses, or an alternative splice form or mutant thereof.

68. The recombinant plasmid or recombinant viral vector of claim 67, wherein the primate polyomavirus agnoprotein gene or large T antigen gene target sequence is also contained in an mRNA produced from a JCV, BKV or SV40 gene.

69. The recombinant plasmid or recombinant viral vector of claim 67, wherein the nucleic acid sequences for expressing the siRNA comprise an inducible or regulatable promoter.

70. The recombinant plasmid or recombinant viral vector of claim 69, comprising a CMV type Pol-II promoter.

71. A pharmaceutical composition comprising a siRNA comprising a pharmaceutically acceptable carrier and an active agent selected from:
(i) a sense RNA strand and an antisense RNA strand, wherein the sense and antisense RNA strands form an RNA duplex, and wherein the sense RNA strand comprises a nucleotide sequence substantially identical to a target sequence of about 19 to about 25 contiguous nucleotides in agnoprotein gene or large T antigen gene mRNA of primate polyomviruses, or an alternative splice form or mutant thereof;
(ii) a recombinant plasmid or recombinant viral vector comprising nucleic acid sequences for expressing an siRNA comprising a sense RNA strand and an antisense RNA strand, wherein the sense and an antisense RNA strands form an RNA duplex, and wherein the sense RNA strand comprises a nucleotide sequence substantially identical to a target sequence of about 19 to about 25 contiguous nucleotides in agnoprotein gene or large T antigen gene mRNA of primate polyomaviruses, or an alternative splice form or mutant thereof, further wherein the nucleic acid sequences for expressing the siRNA comprise a sense RNA strand coding sequence in operable connection with a polyT termination sequence under the control of a human U6 type RNA Pol-III promoter, and an antisense RNA strand coding sequence in operable connection with a polyT termination sequence under the control of a human U6 type RNA Pol-III promoter; or
(iii) a recombinant plasmid or recombinant viral vector comprising nucleic acid sequences for expressing an siRNA in which the sense and antisense strand coding sequences are contained within one contiguous sequence such that a sense RNA strand is followed by a loop followed by an antisense RNA strand in operable connection with a termination sequence.

72. The recombinant plasmid or recombinant viral vector of claim 67 comprising nucleic acid sequences for expressing an siRNA in which the sense and antisense strand coding sequences are contained within one contiguous sequence such that a sense RNA strand is followed by a loop followed by an antisense RNA strand in operable connection with a termination sequence.

73. A method of inhibiting expression of a primate polyomavirus gene wherein the infected cell is human or primate cell, comprising administering to a subject an effective amount of an active agent selected from:
(i) an isolated siRNA comprising a sense RNA strand and an antisense RNA strand, wherein the sense and antisense RNA strands form an RNA duplex, and wherein the sense RNA strand comprises a nucleotide sequence substantially identical to a target sequence of about 19 to about 25 contiguous nucleotides in agnoprotein gene or large T antigen gene mRNA of primate polyomviruses, or an alternative splice form or mutant thereof; or
(ii) a recombinant plasmid or recombinant viral vector comprising nucleic acid sequences for expressing an siRNA comprising a sense RNA strand and an antisense RNA strand, wherein the sense and an antisense RNA strands form an RNA duplex, and wherein the sense RNA strand comprises a nucleotide sequence substantially identical to a target sequence of about 19 to about 25 contiguous nucleotides in agnoprotein gene or large T antigen gene mRNA of primate polyomaviruses, or an alternative splice form or mutant thereof, further wherein the nucleic acid sequences for expressing the siRNA comprise a sense RNA strand coding sequence in operable connection with a polyT termination sequence under the control of a human U6 type RNA Pol-III promoter, and an antisense RNA strand coding sequence in operable connection with a polyT termination sequence under the control of a human U6 type RNA Pol-II promoter;
(iii) a recombinant plasmid or recombinant viral vector comprising nucleic acid sequences for expressing an siRNA in which the sense and antisense strand coding sequences are contained within one contiguous sequence such that a sense RNA strand is followed by a loop followed by an antisense RNA strand in operable connection with a termination sequence.

74. The method of claim 73, wherein the agnoprotein gene or large T antigen gene target sequence of primate polyomaviruses is contained in an mRNA produced from a JCV, BKV or SV40 gene, and wherein the primate polyomavirus gene is a gene from JCV, BKV or SV40.

75. The method of claim 73, wherein two or more pharmaceutical compositions comprising a siRNA comprising a pharmaceutically acceptable carrier and an active agent selected from:
(i) a sense RNA strand and an antisense RNA strand, wherein the sense and antisense RNA strands form an RNA duplex, and wherein the sense RNA strand comprises a nucleotide sequence substantially identical to a target sequence of about 19 to about 25 contiguous nucleotides in agnoprotein gene or large T antigen gene mRNA of primate polyomviruses, or an alternative splice form or mutant thereof;
(ii) a recombinant plasmid or recombinant viral vector comprising nucleic acid sequences for expressing an siRNA comprising a sense RNA strand and an antisense RNA strand, wherein the sense and an antisense RNA strands form an RNA duplex, and wherein the sense RNA strand comprises a nucleotide sequence substantially identical to a target sequence of about 19 to about 25 contiguous nucleotides in agnoprotein gene or large T antigen gene mRNA of primate polyomaviruses, or an alternative splice form or mutant thereof, further wherein the nucleic acid sequences for expressing the siRNA comprise a sense RNA strand coding sequence in operable connection with a polyT termination sequence under the control of a human U6 type RNA Pol-III promoter, and an antisense RNA strand coding sequence in operable connection with a polyT termination sequence under the control of a human U6 type RNA Pol-III promoter; or
(iii) a recombinant plasmid or recombinant viral vector comprising nucleic acid sequences for expressing an siRNA in which the sense and antisense strand coding sequences are contained within one contiguous sequence such that a sense RNA strand is followed by a loop followed by an antisense RNA strand in operable connection with a termination sequence, are administered to the subject, and wherein each pharmaceutical composition administered comprises a nucleotide sequence which is substantially identical to a different primate polyomavirus agnoprotein gene or large T antigen gene mRNA target sequence.

76. The method of claim 73, wherein two or more pharmaceutical compositions comprising a siRNA comprising a pharmaceutically acceptable carrier and an active agent selected from:
(i) a sense RNA strand and an antisense RNA strand, wherein the sense and antisense RNA strands form an RNA duplex, and wherein the sense RNA strand comprises a nucleotide sequence substantially identical to a target sequence of about 19 to about 25 contiguous nucleotides in agnoprotein gene or large T antigen gene mRNA of primate polyomviruses, or an alternative splice form or mutant thereof;
(ii) a recombinant plasmid or recombinant viral vector comprising nucleic acid sequences for expressing an siRNA comprising a sense RNA strand and an antisense RNA strand, wherein the sense and an antisense RNA strands form an RNA duplex, and wherein the sense RNA strand comprises a nucleotide sequence substantially identical to a target sequence of about 19 to about 25 contiguous nucleotides in agnoprotein gene or large T antigen gene mRNA of primate polyomaviruses, or an alternative splice form or mutant thereof, further wherein the nucleic acid sequences for expressing the siRNA comprise a sense RNA strand coding sequence in operable connection with a polyT termination sequence under the control of a human U6 type RNA Pol-III promoter, and an antisense RNA strand coding sequence in operable connection with a polyT termination sequence under the control of a human U6 type RNA Pol-III promoter; or
(iii) a recombinant plasmid or recombinant viral vector comprising nucleic acid sequences for expressing an siRNA in which the sense and antisense strand coding sequences are contained within one contiguous sequence such that a sense RNA strand is followed by a loop followed by an antisense RNA strand in operable connection with a termination sequence, are administered to the subject, and wherein each pharmaceutical composition administered comprises a nucleotide sequence which is substantially identical to a target sequence from an mRNA produced from a JCV, BKV or SV40 gene.

77. The method of claim 73, wherein said active agent is provided to said subject by parenteral administration.

78. A method of inhibiting polyomavirus replication or infection in a subject, comprising administering to a subject an effective amount of an active agent selected from
(i) an isolated siRNA comprising a sense RNA strand and an antisense RNA strand, wherein the sense and antisense RNA strands form an RNA duplex, and wherein the sense RNA strand comprises a nucleotide sequence substantially identical to a target sequence of about 19 to about 25 contiguous nucleotides in agnoprotein gene or large T antigen gene mRNA of primate polyomviruses, or an alternative splice form or mutant thereof;
(ii) a recombinant plasmid or recombinant viral vector comprising nucleic acid sequences for expressing an siRNA comprising a sense RNA strand and an antisense RNA strand, wherein the sense and an antisense RNA strands form an RNA duplex, and wherein the sense RNA strand comprises a nucleotide sequence substantially identical to a target sequence of about 19 to about 25 contiguous nucleotides in agnoprotein gene or large T antigen gene mRNA of primate polyomaviruses, or an alternative splice form or mutant thereof, further wherein the nucleic acid sequences for expressing the siRNA comprise a sense RNA strand coding sequence in operable connection with a polyT termination sequence under the control of a human U6 type RNA Pol-III promoter, and an antisense RNA strand coding sequence in operable connection with a polyT termination sequence under the control of a human U6 type RNA Pol-III promoter; or
(iii) a recombinant plasmid or recombinant viral vector comprising nucleic acid sequences for expressing an siRNA in which the sense and antisense strand coding sequences are contained within one contiguous sequence such that a sense RNA strand is followed by a loop followed by an antisense RNA strand in operable connection with a termination sequence

79. The method of claim 78 for inhibiting JCV, BKV or SV40 replication or infection in a subject.

80. A method of treating a disease associated with JCV, BKV or SV40 infection in a subject, comprising administering to a subject in need an active agent selected from:
(i) an isolated siRNA comprising a sense RNA strand and an antisense RNA strand, wherein the sense and antisense RNA strands form an RNA duplex, and wherein the sense RNA strand comprises a nucleotide sequence substantially identical to a target sequence of about 19 to about 25 contiguous nucleotides in agnoprotein gene or large T antigen gene mRNA of primate polyomviruses, or an alternative splice form or mutant thereof; or
(ii) a recombinant plasmid or recombinant viral vector comprising nucleic acid sequences for expressing an siRNA comprising a sense RNA strand and an antisense RNA strand, wherein the sense and an antisense RNA strands form an RNA duplex, and wherein the sense RNA strand comprises a nucleotide sequence substantially identical to a target sequence of about 19 to about 25 contiguous nucleotides in agnoprotein gene or large T antigen gene mRNA of primate polyomaviruses, or an alternative splice form or mutant thereof, further wherein the nucleic acid sequences for expressing the siRNA comprise a sense RNA strand coding sequence in operable connection with a polyT termination sequence under the control of a human U6 type RNA Pol-III promoter, and an antisense RNA strand coding sequence in operable connection with a polyT termination sequence under the control of a human U6 type RNA Pol-III promoter; or
(iii) a recombinant plasmid or recombinant viral vector comprising nucleic acid sequences for expressing an siRNA in which the sense and antisense strand coding sequences are contained within one contiguous sequence such that a sense RNA strand is followed by a loop followed by an antisense RNA strand in operable connection with a termination sequence.

81. The method of claim 80, wherein the disease associated with JCV infection is cancer or a demyelinating disease; wherein the disease associated with BKV infection is cancer or polyomavirus nephropathy; or wherein the disease associated with SV40 infection is cancer.

82. The method of claim 81, wherein the JCV associated cancer is medulloblastoma or glioblastoma, or the BKV associated cancer is prostate cancer or of urogenital origin, or the SV40 associated cancer is mesothelioma.


SEQUENCE LISTING (9 sequences)

SEQ ID NO: 1 - DNA, 216 bp, JC virus
atggttcttcgccagctgtcacgtaaggcttctgtgaaagttagtaaaacctggagtggaactaaaaaaagagctcaaaggattttaatttttttgttagaatttttgctggacttttgcacaggtgaagacagtgtagacgggaaaaaaagacagagacacagtggtttgactgagcagacatacagtgctttgcctgaaccaaaagctacatag

SEQ ID NO: 2 - DNA, 216 bp, JC virus
atggttcttcgccagctgtcacgtaaggcttctgtgaaagttagtaaaacctggagtggaactaaaaaaagagctcaaaggattttaatttttttgttagaatttttgctggatttttgcacaggtgaagacagtgtagacgggaaaaaaagacagaaacacagtggtttgactgagcagacatacagtgctttgcctgaaccaaaagctacatag

SEQ ID NO: 3 - DNA, 216 bp, JC virus; identical to SEQ ID NO: 1

SEQ ID NO: 4 - DNA, 216 bp, JC virus
atggttcttcgccagctgtcacgtaaggcttctgtgaaagttagtaaaacctggagtggaactaaaaaaagagcccaaaggattttaatttttttgttagaatttttgctggatttttgcacaggtgaagacagtgtagacgggaaaaaaagacagaaacacagtggtttgactgagcagacatacagtgctttgcctgaaccaaaagctaaatag

SEQ ID NO: 5 - DNA, 216 bp, JC virus
atggttcttcgccagctgtcacgtaaggcttctgtgaaagttagtaaaacctggagtggaactaaaaaaagagctcaaaggattttaatttttttgttagaatttttgctggacttttgcacaggtgaagacagagtagacgggaaaaaaagacagaaacacagtggtttgactgagcagacatacagtgctttgcctgaaccaaaagctacatag

SEQ ID NO: 6 - DNA, 216 bp, JC virus
atggttcttcgccagctgtcacgtaaggcttctgtgaaagttagtaaaacctggagtggaactaaaaaaagagctcaaaggattttaatttttttgttagaatttttgctggacttttgcacaggtgaagacagtgtagacgggaaaaaaagacagaaacacagaggtttgactgagcagacatacagtgctttgcctgaaccaaaagctacatag

SEQ ID NO: 7 - DNA, 225 bp, BK virus
atgttttgcgagcctaagaatcttgtggttttgcgccagctgtcacgacaagcttcagtgaaagttggtaaaacctggactggaactaaaaaaagagctcagaggatttttatttttattttagagcttttgctggaattttgtagaggtgaagacagtgtagacgggaaaaacaaaagtaccactgctttacctgctgtaaaagactctgtaaaagactcctag

SEQ ID NO: 8 - DNA, 201 bp, BK virus
atggttctgcgccagctgtcacgacaagcttcagtgaaagttggtaaaacctggactggaacaaaaaaaagagctcagaggatttttatttttattttagagcttttgctggaattttgtagaggtgaagacagtgtagacgggaaaaacaaaagtaccactgctttacctgctgtaaaagactctgtaaaagactcctag

SEQ ID NO: 9 - DNA, 189 bp, SV40
atggtgctgcgccggctgtcacgccaggcctccgttaaggttcgtaggtcatggactgaaagtaaaaaaacagctcaacgcctttttgtgtttgttttagagcttttgctgcaattttgtgaaggggaagatactgttgacgggaaacgcaaaaaaccagaaaggttaactgaaaaaccagaaagttaa